SEQUENCE LISTING (1 sequence)

SEQ ID NO: 1 - protein, 12 aa, synthetic construct
  REGION 1..12: note = Synthetic - Exemplary targeting peptide
WHWLPNLRHYAS

(19) United States
(12) Patent Application Publication
(10) Pub. No.: US 2026/0256698 A1
(43) Pub. Date: Sep. 3, 2026

(54) REHYDRATABLE, MORPHOLOGICALLY DIVERSE NANOCARRIER POWDERS

(71) Applicant: Northwestern University, Evanston, IL (US)

(72) Inventors: Evan Alexander Scott, Evanston, IL (US); Michael Patrick Vincent, Evanston, IL (US); Sharan Kumar Reddy Bobbala, Evanston, IL (US)

(21) Appl. No.: 19/382,963

(22) Filed: Nov. 7, 2025

Related U.S. Application Data
(63) Continuation of application No. 17/807,372, filed on Jun. 16, 2022, now abandoned.
(60) Provisional application No. 63/202,563, filed on Jun. 16, 2021.

(51) Int. Cl. A61K 9/16

(52) U.S. Cl. CPC A61K 9/1647; A61K 9/1623; A61K 9/1641

(57) ABSTRACT

Provided herein are rehydratable powdered formulations of nanocarriers that can be used to encompass hydrophobic or hydrophilic cargo. The formulations can be used for medicinal, agricultural, and research applications. Methods of making the formulations are also provided.

### CROSS-REFERENCE TO RELATED APPLICATIONS

The present application is a continuation of U.S. patent application Ser. No. 17/807,372 filed Jun. 16, 2022, which claims priority under 35 U.S.C. § 119 (e) to U.S. Provisional Patent Application No. 63/202,563 filed Jun. 16, 2021, each of which are hereby incorporated by reference in their entireties herein.

### STATEMENT REGARDING FEDERALLY SPONSORED RESEARCH

This invention was made with government support under AI145345, HL132390, and AI137932, awarded by the National Institutes of Health, and 1453576, awarded by the National Science Foundation. The government has certain rights in the invention.

### SEQUENCE LISTING

The XML file named “393044_7006US2_SequenceListing.xml” created on Nov. 7, 2025, comprising 2,314 bytes, is hereby incorporated by reference in its entirety.

### BACKGROUND OF THE INVENTION

The broad use of self-assembled nanocarriers (e.g., liposomes, micelles, and polymersomes) in medical, research, and commercial applications is burdened by the large number of resources and skills needed to prepare stable, reliable formulations. Nanocarrier formulations are commonly prepared using emulsification¹, cosolvent evaporation², thin-film rehydration^{3,4}, or flash nanoprecipitation⁵⁻¹⁰ techniques. These methods utilize multi-step procedures, harsh conditions (e.g., organic solvents, sonication), and/or expensive equipment, as well as significant allocations of time and/or financial resources. Aside from the more commonly recognized concerns, such as the exposure of biologic cargo (i.e., proteins, nucleic acids, etc.) to harsh organic solvents, the use of these methods to fabricate nanocarriers often takes a trained professional multiple days to prepare stable, monodisperse formulations. This is problematic since endpoint users of nanocarriers usually do not have the training or time to prepare formulations for their intended applications. Furthermore, many nanocarrier platforms are formulated as aqueous suspensions that become unstable in storage over time, largely due to oxidation and hydrolysis. These stability issues lead to various problems, ranging from agglomeration to cargo leakage, rendering stored formulations unusable shortly after the point of creation.

Thus, there is a need for a less expensive, less harsh, and less complex fabrication method for polymeric nanocarriers. There is also a need for polymeric nanocarrier formulations that are stable during long-term storage.

### SUMMARY OF THE INVENTION

In a first aspect, the present disclosure provides rehydratable powdered formulations comprising (a) a polymeric amphiphile and (b) a carbohydrate.

In a second aspect, the present disclosure provides methods of producing an aqueous suspension of a nanocarrier from a rehydratable powdered formulation that comprises a polymeric amphiphile and a carbohydrate. The methods comprise (a) contacting the formulation with a liquid to form a rehydrated mixture and (b) vortexing the rehydrated mixture.

In a third aspect, the present disclosure provides methods of producing a rehydratable powdered formulation. The methods comprise (a) dissolving a polymeric amphiphile in a first organic solvent to form a solution, (b) adding to the solution a carbohydrate to make a mixture, and (c) evaporating the organic solvent from the mixture in a vacuum desiccator to form the rehydratable powder comprising a polymeric amphiphile and carbohydrate.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 shows formation of morphologically diverse nanocarriers upon rehydration with aqueous media. (A) Schematic of the formulation strategy and the resulting PEG-b-PPS nanocarrier morphologies. Powders consisting of PEG-b-PPS polymer and mannitol carbohydrate are prepared. Upon adding water and vortexing, the nanocarriers rapidly self-assemble into the expected morphology based on the hydrophilic (PEG) weight fraction (f_{PEG}) range of the polymer. Abbreviations: polymersome (PS); filomicelle (FM); micelle (MC). (B) Transmission electron microscopy (TEM) of negatively stained rehydrated nanocarriers (30,000×; scale bar=200 nm). Orange arrows point to MC. The theoretical cargo loading compatibility is depicted below each micrograph and is experimentally demonstrated in the Examples.

FIG. 2 shows results of powder characterization by scanning electron microscopy and X-ray diffraction. (A) Scanning electron microscopy (SEM) micrographs of mannitol (M), mannitol+ micelle polymer (M+MC), mannitol+filomicelle polymer (M+FM), and mannitol+polymersome polymer (M+PS) powders. Top panel=300× magnification (scale bar=100 μm). Bottom panel=1500× magnification (scale bar=30 μm). (B) Powder x-ray diffraction analysis of mannitol alone (control) and mannitol in complex with PEG-b-PPS polymers in powdered form.

FIG. 3 demonstrates that rehydrated nanocarriers are non-toxic and are endocytosed by macrophages at a rate that is dependent on morphology. (A, B) Analysis of macrophage (A) viability and (B) median fluorescence intensity (MFI) after incubation with nanocarriers at the specified polymer concentration for 2 h. For the viability studies in (A), PBS (white bar) and short duration exposure to 70% ethanol (grey bar) were included as controls. Significant differences in cell viability versus toxicity control (70% ethanol) were determined by ANOVA with Tukey's multiple comparisons test and a 5% significance level. ****p<0.0001. (C) Flow cytometric analysis of nanocarrier uptake after pre-treating cells with PBS (no inhibitor), 50 μM CytD (phagocytosis inhibitor), or 50 μM CPZ (clathrin-mediated endocytosis inhibitor). Nanocarriers were administered at a 1.0 mg/mL polymer concentration for endocytosis inhibitor studies. For (B,C) significant differences were determined by two-way ANOVA with Tukey's multiple comparisons test and a 5% significance level. Comparisons within concentration groupings: ****p<0.0001, ***p≤0.0005, **p<0.005, *p<0.05. For all analyses, cellular viability and nanocarrier uptake studies used a 2 h nanocarrier incubation period. Cells were cultured at 37° C., 5% CO₂. The mean±s.e.m. (n=3) is displayed.

FIG. 4 demonstrates the targeting functionality of rehydrated PEG-b-PPS nanocarriers displaying lipid-anchored targeting peptides. (A) Powders were prepared to consist of PEG-b-PPS, mannitol, and lipid-anchored targeting peptide. Nanocarriers self-assembled upon rehydration. MC (top), FM (middle), and PS (bottom) displaying a linear targeting peptide is displayed. Cryo-TEM micrographs were acquired at 10,000×. Scale bar=100 nm. (B, C) Demonstration of targeted micellar delivery of hydrophobic cargo (B) and targeted vesicular delivery of biological cargo (C). MC and PS nanocarriers were prepared with displayed cyclic RGD peptides at 1% and 5% molar ratios, respectively. The fold change in uptake by primary human umbilical vein endothelial cells (HUVECs) was quantified as the median fluorescence intensity (MFI) above untargeted control. The mean±s.e.m. is displayed. Statistically significant differences in fold changes were determined between the two molar ratio groups using an unpaired t test and a 5% significance level. **p<0.01.

FIG. 5 shows a morphological characterization of biologic-loaded polymersomes and bioactivity assessment of enzymatic cargo. (A) TEM of negatively stained PS loaded with Dex-TMR (left) or AP enzyme (right) following rehydration. (B) Characterization of AP enzyme activity in unfiltered or SEC-filtered AP-loaded PS formulations following cargo release with 1% triton. BCIP/NBT substrate was administered and the activity of AP enzyme monitoring with time by measuring the absorbance of 630 nm light. (C) Cell viability of RAW 264.7 macrophages treated with polymersomes ([polymer]=1.0 mg/mL) loaded with Dex-TMR hydrophilic tracer. Cells treated with PBS or 70% ethanol (EtOH) were included as controls. Significant differences in cell viability versus the EtOH treatment group were determined by ANOVA with Dunnett's multiple comparisons test and a 5% significance level. (D) Median fluorescence intensity (MFI) of macrophages treated with PS ([polymer]=1.0 mg/mL) encapsulating Dex-TMR. Where specified, cells were pre-treated with 50 μM CytD or 50 μM CPZ inhibitor prior to PS administration. Statistically significant differences in MFI were determined by ANOVA with Tukey's multiple comparisons test and a 5% significance level. The mean±s.e.m. (n=3) is presented in all cases. For all statistical tests, ****p<0.0001, *p<0.05.

FIG. 6 shows powdered formulations of a commercially available polymer amphiphile and characterization of nanocarriers that self-assemble after rehydration. (A) Illustration and image of powder formed from mannitol and commercially available PEG-b-polystyrene polymer. (B) SEM micrographs of powders (left=300× magnification; right=1500× magnification). (C) Powder XRD of mannitol (control; top) and mannitol coated with PEG-b-polystyrene (bottom). (D) TEM micrograph of rehydrated PEG-b-polystyrene micelles (30,000× magnification; Scale bar=200 nm). (E-G) Cellular viability and uptake studies performed with PEG-b-polystyrene nanocarriers and RAW 264.7 macrophages. (E) Viability of macrophages after 2 h treatment with PEG-b-polystyrene nanocarriers dosed at the specified polymer concentration. PBS- or 70% ethanol (EtOH)-treated cells were included as controls. Significant differences in cell viability versus the EtOH-treated cells were determined by ANOVA with Dunnett's multiple comparisons test and a 5% significance level. (F) Median fluorescence intensity (MFI) of macrophages treated with PEG-b-polystyrene nanocarriers (0.25-1.0 mg/mL polymer concentration) for 2 h. (G) Cellular MFI after pre-treatment with CytD (50 mM) or CPZ (50 mM) endocytosis inhibitors prior to nanocarrier administration. For (F, G), significant differences in cellular uptake were determined by ANOVA with Tukey's multiple comparisons test and a 5% significance level. The mean±s.e.m. (n=3) is displayed in (E-G).

FIG. 7 shows slurries of mannitol and PEG-b-PPS polymers in DCM prior to drying and powder formation. A 3:1 ratio of carbohydrate: polymer was used to prepare each slurry.

FIG. 8 shows the percent transmittance of rehydrated PEG-b-PPS nanocarrier formulations of diverse morphologies. The transmittance of 405 nm light was determined for rehydrated micelles (MC), filomicelles (FM), and polymersomes (PS).

FIG. 9 shows the results of small angle x-ray scattering (SAXS) of rehydrated vesicular polymersomes. SAXS was performed using synchrotron radiation at Argonne National Laboratory. A vesicle model was fit to the scattering profile of rehydrated polymersomes (model fit: X²=3.9; radius=47 nm; thickness=7.8 nm).

FIG. 10 shows a TEM micrograph of rehydrated DiD-loaded PS suspension after 1 year storage at room temperature. The TEM micrograph was acquired at 30,000×. Scale bar=200 nm.

FIG. 11 shows PEG-b-PPS PS formed after rehydration from polymer-carbohydrate powders formed in different solvents. Powders were prepared by vacuum desiccation of acetone-, chloroform-, or THF-based slurries. TEM micrographs of the unpurified PS following rehydration are displayed (30,000×; scale bar=200 nm).

FIG. 12 shows PEG-b-PPS PS formed after rehydration from polymer-carbohydrate powders formed using different carbohydrates. Powders were prepared by vacuum desiccation of slurries composed of PEG-b-PPS PS polymer and the specified carbohydrate (glucose, lactose, or trehalose). TEM micrographs of the unpurified PS following rehydration are displayed (30,000×; scale bar=200 nm).

FIG. 13 shows SEM micrographs of alternative carbohydrates and corresponding polymer-carbohydrate powders. SEM micrographs at low magnification (top row) or high magnification (middle row) of free glucose, lactose, or trehalose carbohydrates are displayed. Micrographs of polymer-coated carbohydrates acquired at high magnification (300× or 450×) are displayed on the bottom row.

FIG. 14 shows images of DiI-loaded PEG-b-PPS nanocarriers of diverse morphology after rehydration with PBS. Dried powders consisting of mannitol carbohydrate, PEG-b-PPS polymer, and DiI drug were rehydrated with PBS to a final polymer concentration of 10 mg/ml and were briefly vortexed to mix.

FIG. 15 shows the results of an assessment of cell viability using the MTT assay. RAW 264.7 macrophages were incubated with rehydrated nanocarriers at the indicated concentration (in mg/mL), or with mannitol (Mann; 3 mg/mL) or Carboplatin (CB; 1 mg/mL) for 24 h at 37° C. The percentage of viable cells is displayed. Significant differences in cell viability versus the CB toxicity group was determined by ANOVA with Dunnett's multiple comparisons test and a 5% significance level. **p<0.005.

FIG. 16 shows the calibration curve used to determine the concentration of loaded alkaline phosphatase enzyme. The absorbance of 660 nm light of an alkaline phosphatase concentration series was measured in triplicate and the data were fit via a simple linear regression model (A₆₆₀=0.08256c+0.1063, where A₆₆₀ is the absorbance of 660 nm light and c is the concentration (in mg/mL); r²=0.9864).

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

The present disclosure provides a rehydratable powdered formulation, specifically a rehydratable powder formulation of self-assembling polymeric nanocarriers. These nanocarriers can be used in medical, basic research, and commercial applications and can have diverse morphologies (micelles, filamentous micelles, and polymersomes). In the Examples, the inventors demonstrate efficient loading of these nanocarriers with hydrophobic and hydrophilic cargoes for targeted delivery to cells. Powders that incorporate lipid-anchored targeting peptides (i.e., both linear and cyclic) displayed these peptides at the surface without disrupting nanocarrier self-assembly. The novel powdered formulations of polymeric nanocarriers described herein include formulations of any composition capable of assembling into diverse nanostructure morphologies. This technology will enable the widespread use of polymeric nanomaterials in academic and commercial pursuits and will provide new strategies for the storage and transport of nanomaterial-based formulations.

The formulated powders of the present invention are highly stable, achieving rehydration into monodisperse nanocarriers following over 6 months of storage. Diverse drug cargoes were efficiently encapsulated during rehydration, including hydrophobic small molecules for micellar morphologies. Individual and concurrent loading of hydrophobic and hydrophilic molecules for vesicular morphologies were also accomplished. Rehydrated polymersomes are shown to load hydrophilic biological macromolecules, and encapsulated enzymes retain bioactivity. Furthermore, the inventors demonstrate that inclusion of lipid-anchored ligands in powder form permits the surface-display of targeting ligands and enhances target cell uptake, thereby extending this technology to targeted drug delivery applications. Their powder-based formulation strategy is extendable to other polymer amphiphiles, including PEG-b-polystyrene and PEG-b-polycaprolactone. The formulated nanotechnologies described herein are highly modular, require minimal preparation, remain stable in ambient long-term storage (bypassing cold chain requirements), and will enable their use in medicine (human and veterinary), research, and commercial applications from cosmetics to agriculture.

The present invention also provides methods of producing aqueous suspensions of polymeric nanocarriers from the rehydratable powdered formulation, as well as methods of producing the rehydratable powdered formulation from carbohydrates and polymeric amphiphiles. Use of these hydratable powder formulations for targeted deliver is also contemplated.

### Formulations:

In a first aspect, the present disclosure provides rehydratable powdered formulations that comprise or consists of (a) a polymeric amphiphile and (b) a carbohydrate.

The term “rehydratable” is used herein to refer to a substance that is capable of being rehydrated (i.e., restored to a fluid state following dehydration).

The term “powdered” is used herein to refer to a substance that is in the form of a powder. A “powder” is a dry, bulk solid composed of many very fine particles.

A “polymeric amphiphile” is an amphiphilic copolymer comprised of sub-units or monomers that have different hydrophilic and hydrophobic characteristics. Typically, these sub-units are present in groups of at least two, comprising a block of a given character, such as a hydrophobic or hydrophilic block. Depending on the method of synthesis, these blocks could be of all the same monomer or contain different monomer units dispersed throughout the block, but still yielding blocks of the copolymer with substantially hydrophilic and hydrophobic portions. These blocks can be arranged into a series of two blocks (diblock) or three blocks (triblock), or more, forming the backbone of a block copolymer. In addition, the polymer chain may have chemical moieties covalently attached or grafted to the backbone. Such polymers are graft polymers. Block units making up the copolymer can occur in regular intervals, or they can occur randomly making a random copolymer. In addition, grafted side chains can occur at regular intervals along the polymer backbone or randomly making a randomly grafted copolymer. The ratio of the hydrophobic to hydrophilic blocks of the copolymer will be selected such that the soluble and insoluble components are balanced and suitable aggregation for the desired architectures. In some embodiments, the polymeric amphiphile included in the formulation is poly(ethylene glycol)-block-poly(propylene sulfide) (PEG-b-PPS) copolymer, PEG-b-polystyrene polymer, or PEG-b-polycaprolactone polymer.

The polymeric amphiphile used with the present invention may include chemical modifications or end caps. Suitable chemical modifications and end caps include, but are not limited to, thiol, benzyl, pyridyl disulfide, phthalimide, vinyl sulfone, aldehyde, acrylate, maleimide, and n-hydroxysuccinimide groups. The chemical modification of the copolymer may add a charged residue to the polymer or may be used to otherwise functionalize the polymer. The ability to functionalize the polymer allows for suitable reactivities for further modification, if required or wanted.

In some embodiments, the polymeric amphiphile is PEG-b-PPS copolymer. Suitable preparations of PEG-b-PPS nanocarriers can be prepared via known methods. See, e.g., Du et al. (Nat Commun 29; 11 (1): 48962019, 2020), Du et al. (J Control Release 282:90-100, 2018), and Yi et al. (ACS Nano 10 (12): 11290-11303, 2016) each of which are incorporated herein by reference in their entirety. In some embodiments, the PEG-b-PPS copolymer has a PEG weight fraction range of 0.2-0.6. The PEG weight fraction for a block copolymer is the ratio of the PEG block molecular weight (MW) to MW of the hydrophobic block (e.g., MW PEG/MW PPS).

In other embodiments, the polymeric amphiphile is a PEG-b-polystyrene polymer or a PEG-b-polycaprolactone polymer, which can be obtained from commercial vendors such as Polymer Source, Inc.

In some embodiments, the polymeric amphiphile has a glass transition temperature lower than −30° C. A “glass transition temperature” is the temperature where the polymer changes from a rigid glassy material to a soft (not melted) material.

In some embodiments, the polymeric amphiphile coats the carbohydrate. The term “coats” refers to associating with a surface of a material or compound via covalent or noncovalent interactions.

A “carbohydrate” is a biomolecule consisting of carbon (C), hydrogen (H) and oxygen (O) atoms. Typically, carbohydrates have a hydrogen-oxygen atom ratio of 2:1 have the empirical formula Cₘ(H₂O)ₙ, wherein m may or may not be different from n. However, not all carbohydrates conform to this precise stoichiometric definition. Suitable carbohydrate for use in the formulations of the present invention include, for example, mannitol, trehalose, lactose, or glucose.

In some embodiments, the polymeric amphiphile and the carbohydrate has a mass ratio of 1:3. A “mass ratio”, often called a “percent composition by mass,” describes the proportions of elements included in a mixture in terms of their masses.

In some embodiments, the rehydratable powdered formulation further comprises or consists of a ligand comprising a peptide, a PEG spacer, and a lipid anchor (i.e., a lipid anchored ligand). The inclusion of a lipid-anchored ligand in the formulation permits the surface-display of the ligand. Such ligands can be used to enhance target cell uptake, making this technology useful for targeted drug delivery applications.

As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a portion of naturally occurring protein or may be artificial). In preferred embodiments, the peptide binds to a receptor present on a target cell. Thus, in some embodiments, the peptide is capable of targeting the nanocarrier to target cells, allowing for specific targeting of the cargo. In the Examples, the inventors used the targeting peptide arginine-glycine-aspartate (RGD). RGD binds to integrin receptors, which are found on various types of tumor endothelium cells.

Other examples include peptide motifs that bind to other cell receptors, proteins or cellular components, including, but not limited to WHWLPNLRHYAS for FLT4 receptors, KTTKQSFDLSVKAQYKKKNKHK for CD11c receptors, (KKEEE) 3K YIGSR for laminin receptors, KRSR for heparan-sulfate, for PKKKRKV the nucleus, MLSLRQSIRFFKPATRTLCSSRYLL for the mitochondria, and MMSFVSLLLVGILFWATEAEQLIKCEVFQ for the endoplasmic reticulum and include sequences with at least 80% identity, 85% identity, 90% identity, 95% identity, 98% identity, 100% identity to these protein sequences. This application is not limited by any of these peptides, as there are hundreds of potential peptides that could be used and recognized by one skilled in the art.

In some embodiments, the peptide and the polymeric amphiphile has a molar ratio of 1%-5%. A “molar ratio” is the ratio between the amounts (in moles) of any two substances involved in a chemical reaction.

As used herein, the term “PEG spacer” refers to a formulation of polyethylene glycol (PEG) comprising from about 6 to 20 units. A unit of PEG comprises —(O—CH₂—CH₂)— and in some embodiments the PEG spacer has a formula “—HN—C(O)—CH₂—(O—CH₂—CH₂)ₙ—NH—C(O)—,” wherein one end is attached to the peptide and the other end is attached to the carboxylic acid of the lipid anchor, and wherein “n” is an integer of 6-20. As used herein, the term “unit” refers to the number “n” in the formula of PEG spacer. In some embodiments, the PEG spacer has 6 units.

As used herein, a “lipid anchor” is a lipid that is conjugated to peptide and is able to embed within a cell membrane. The lipid anchor is used to anchor the peptide to a cell surface. Any carbon chain, lipid, fatty acid as well as cholesterol variant with a hydrophobic Log P (>0) could also be used. Examples include, but are not limited to, for example, farnesyl, palmitate and myristate, among many others. In some embodiments of the present invention, the lipid anchor is derived from palmitoleic acid.

The rehydratable powered formulations described herein may further comprise a cargo. As used herein, the term “cargo” is used to refer to any molecule to be loaded into the nanocarriers formed from the formulation. The cargo may be hydrophobic (i.e., repelled by water) or hydrophilic (i.e., attracted to water).

In some embodiments, the rehydratable powdered formulation comprises a hydrophobic cargo. Suitable hydrophobic cargo that can be included in the rehydratable powdered formulation or loaded into the nanocarriers of the present invention include, but are not limited to, hydrophobic dyes or drugs.

In other embodiments, the rehydratable powdered formulation comprises a hydrophilic cargo. Suitable hydrophilic cargo that can be loaded into the nanocarriers of the present invention include, but are not limited to, RNA, DNA, plasmids, peptides, antibodies, proteins, fluorophores, carbohydrates, small molecule drugs, water soluble synthetic polymers, and combinations thereof.

In some embodiments, the rehydratable powdered formulation is stable at room temperature for 6 months or more, alternatively 1 year or more. Stable is defined as being able to maintain the chemical and/or structural characteristics for a specific period of time. As used herein, “room temperature” is 20-22° C. In some embodiments, the rehydratable powdered formulation is stable at room temperature for at least 8 months. In some embodiments, the rehydratable powdered formulation is stable at room temperature for at least 12 months.

### Methods for Producing an Aqueous Suspension of a Nanocarrier:

In a second aspect, the present disclosure also provides methods of producing an aqueous suspension of a nanocarrier from a rehydratable powdered formulation comprising a polymeric amphiphile and a carbohydrate. The methods comprise (a) contacting the formulation with a liquid to form a rehydrated mixture and (b) vortexing the rehydrated mixture.

As used herein, an “aqueous suspension” is a mixture in which fine particles are suspended in a fluid.

The term “vortexing” refers to a process by which a liquid is mixed using a vortex mixer, i.e., a simple device used commonly in laboratories to mix small volumes of liquid.

As used herein, a “nanocarrier” is a nanomaterial that comprises a complex or vesicular nanoarchitecture and is capable of delivering a cargo to specific cells within a subject. A nanocarrier may encompass the cargo or the cargo may be incorporated into the nanocarrier itself.

In some embodiments, the nanocarrier is a polymersome, micelle, or filomicelle. “Polymersomes” are a class of artificial vesicle nanocarriers that are composed of amphiphilic synthetic block copolymers and have an aqueous core. “Micelles” are roughly spherically shaped groupings of amphiphilic molecules contained in a liquid. “Filomicelles” are filamentous micelles of high aspect ratio (>1) cylindrical structure composed of block copolymer amphiphiles. Micelle and filomicelle nanocarriers are typically smaller (e.g., less than 50 nm) than polymersomes and have a hydrophobic/lipophilic core and a hydrophilic exterior.

In some embodiments, the polymeric amphiphile is PEG-b-polycaprolactone and the method further comprises a step following step (a), the step comprising heating the rehydrated mixture at 60° C. in a water bath (i.e., a container filled with heated water).

Suitable liquids for hydrating the powdered formulation are known in the art and include, diluents, including pharmaceutical diluents. In some embodiments, the liquid is water. In some embodiments, the liquid is saline or phosphate-buffered saline (PBS).

In some embodiments, the liquid further comprises a hydrophilic cargo. In some embodiments, the hydrophilic cargo is selected from an RNA, DNA, plasmid, peptide, protein, antibody, fluorophore, carbohydrate, small molecule drug, water soluble synthetic polymer, and combinations thereof.

In some embodiments, the nanocarrier is a polymersome and has a loading efficiency of 10%-20% of the hydrophilic cargo. In some embodiments, the loading efficiency is from 11% to 19%, or from 12% to 18%, or from 13% to 17%, or from 14% to 16%.

Loading efficiency is defined as the ratio of the amount of drug in the assembled nanoparticle to the total amount of drug applied during formulation of the nanoparticles.

In some embodiments, the powdered formulation used in the method further comprises a hydrophobic cargo and/or a ligand.

A “ligand” is a molecule that binds to another (usually larger) molecule. A ligand that is displayed on the surface of a nanocarrier can be used to target the nanocarrier to any desired cell type. In some embodiments, the ligand comprises an antibody, antibody fragment, aptamer, or peptide. In some embodiments, the ligand comprises (a) an antibody, antibody fragment, aptamer, or peptide, (b) a PEG spacer, and (c) a lipid anchor. The lipid anchor may allow the antibody, antibody fragment, aptamer, or peptide to be displayed on the nanocarrier surface.

In some embodiments, the nanocarrier has a loading efficiency of greater than 95% of the hydrophobic cargo and/or the ligand. In some embodiments, the nanocarrier has a loading efficiency of greater than 96%, or greater than 97%, or greater than 98%, or greater than 99%, of the hydrophobic cargo and/or the ligand.

In some embodiments, the nanocarrier is a polymersome and is loaded with at least one hydrophilic and at least one hydrophobic cargo.

In some embodiments of the present invention, the rehydrated mixture has a concentration of 4%-8% (w/v %) of nanoparticles.

In some embodiments, the aqueous suspension of the nanocarrier is stable at room temperature for 6 months or more. In some embodiments, the aqueous suspension of the nanocarrier is stable at room temperature for at least 8 months. In some embodiments, the aqueous suspension of the nanocarrier is stable at room temperature for at least 12 months.

In some embodiments, the nanocarrier is non-toxic to an immune cell, meaning that it does not negatively impact the viability of the immune cell.

Methods of producing a rehydratable powdered formulation:

In a third aspect, the present disclosure provides methods of producing a rehydratable powdered formulation comprising a polymeric amphiphile and carbohydrate. The methods comprise (a) dissolving a polymeric amphiphile in a first organic solvent to form a solution, (b) adding to the solution a carbohydrate to make a mixture, and (c) evaporating the organic solvent from the mixture in a vacuum desiccator to form the rehydratable powder comprising a polymeric amphiphile and carbohydrate.

An “organic solvent” is a carbon-based substance that is used to dissolve another substance. The term “dissolve” or “dissolving” refers to a process in which a solid substance is incorporated into a liquid to form a solution. In some embodiments of the present invention, the organic solvent is selected from a group consisting of dichloromethane, acetone, chloroform, and tetrahydrofuran.

The term “evaporate” or “evaporating” refers to a process in which a substance transitions from its liquid state to its gaseous state at a temperature below the substance's boiling temperature. Evaporation can be accomplished efficiently using a desiccator. A “desiccator” is a sealable vessel containing desiccants that is used for drying materials. Vacuum desiccators include a stopcock through which a vacuum can be applied for rapid evacuation of air and moisture.

In some embodiments of the present invention, the method further comprises a step following step (b), the step comprising adding a solution comprising a hydrophobic cargo and/or a ligand and a second organic solvent to the mixture prior to step (c).

### Methods of Using the Rehydratable Powdered Formulations:

The present disclosure also contemplates methods of targeting cells using the rehydratable, powdered formulations provided herein. In some embodiments, the formulation comprises a cargo. In some embodiments, the formulation comprises a ligand that specifically targets a particular cell type. The nanoparticles can be designed to target any cell surface molecule, including, proteins, carbohydrates, lipids or nucleic acids. Ligands can be associated with both normal as well as diseases tissue associated with cancer and viral infections.

The use herein of the terms “including,” “comprising,” or “having,” and variations thereof, is meant to encompass the elements listed thereafter and equivalents thereof as well as additional elements. Embodiments recited as “including,” “comprising” or “having” certain elements are also contemplated as “consisting essentially of” and “consisting of” those certain elements.

As used herein, “about” means within 5-10% of a stated concentration range or within 5-10% of a stated number.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit's interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Introduction

Nanocarrier formulations are commonly prepared using emulsification¹, cosolvent evaporation², thin-film rehydration^{3,4}, or flash nanoprecipitation⁵⁻¹⁰ techniques. These methods utilize multi-step procedures, harsh conditions (e.g., organic solvents, sonication), and/or expensive equipment, as well as significant allocations of time and/or financial resources. Aside from the more commonly recognized concerns, such as the exposure of biologic cargo (i.e., proteins, nucleic acids, etc.) to harsh organic solvents, the use of these methods to fabricate nanocarriers often takes a trained professional multiple days to prepare stable, monodisperse formulations. This is problematic since endpoint users of nanocarriers usually do not have the training or time to prepare formulations for their intended applications. Furthermore, many nanocarrier platforms are formulated as aqueous suspensions that become unstable in storage over time, largely due to oxidation and hydrolysis. These stability issues lead to various problems, ranging from agglomeration to cargo leakage, rendering stored formulations unusable shortly after the point of creation.

As compared to traditional aqueous suspensions, rehydratable powders offer useful solutions to each of the aforementioned issues. The powder form is scalable, can be stored for prolonged time periods at room temperature, is compatible with sterilization via gamma irradiation, requires minimal time and skill from the endpoint user, and bypasses concerns involving the exposure of protein cargo to conditions capable of irreversible denaturation and a corresponding loss of bioactivity. Powder forms of self-assembling lipid-based platforms, such as liposomes¹¹⁻¹³ and niosomes^{14,15}, have been developed by leveraging the nanocarrier-stabilizing effects of carbohydrates. However, formulations that employ analogous strategies have not been developed for polymeric self-assembling nanocarriers to date. Here, we present rehydratable powders that successfully form polymeric self-assembled nanocarriers of diverse morphology. Polymeric nanocarriers bypass many of the disadvantages of their lipid-based counterparts, such as pro-liposomal powders¹⁶. For instance, lipid-based systems require multiple additional components (phospholipid mixtures, cholesterol, etc.) to optimize self-assembly and nanocarrier integrity, are confined to spherical morphologies, and suffer from both cargo leakage and hydrolysis in storage. These issues can be overcome, to some degree, by the lyophilization of formed nanocarriers (where compatible). However, lyophilized nanocarriers have their own set of disadvantages, including the choice of a suitable cryoprotectant, compromised nanostructure integrity, and cargo leakage issues after rehydration.

In contrast, the rehydratable polymeric nanocarriers developed herein are minimalistic. The successful formation of stable nanocarriers depends only on the ratio of self-assembling polymer amphiphile and carbohydrate additive. Furthermore, the morphological diversity of our rehydratable polymeric systems has broad utility for medical and commercial applications, including differential loading and cargo release profiles, and provides a set of drug delivery vehicles with distinct circulation times as well as biodistributions at the organ- and cellular-levels^{3,17,18}. Our formulation strategies simultaneously address various issues that are unique to certain morphologies of polymeric nanocarriers. For example, spherical micelle, and cylindrical/filamentous micelle morphologies exhibit stability concerns in storage, resulting in nanocarrier disassembly, cargo loss, and/or cargo aggregation with time. By developing powdered forms, nanocarriers can be self-assembled at the time of use, eliminating issues arising from storage, handling, or rehydration of already formed nanocarriers (e.g., lyophilized powders). Importantly, the ease of preparation for this methodology will extend the utility of polymeric nanocarriers to a broad community of scientists, engineers, and clinicians.

### Results

### Powder Formulation and Characterization of Nanocarrier Self-Assembly Upon Rehydration

Poly(ethylene glycol)-b-poly(propylene sulfide) (PEG-b-PPS) diblock copolymers¹⁹ offer a versatile stimuli-responsive^{3,19-22} drug delivery platform that is capable of self-assembling into morphologically diverse nanocarriers, including micelle (MC), cylindrical micelle (filomicelle; FM), and vesicular polymersome (PS) morphologies^{3,9,18,23}. Furthermore, PEG-b-PPS nanocarriers are non-inflammatory²⁴ and non-toxic in non-human primates²⁵. Importantly, the low glass transition temperature of the PPS block (<-30° C.) 26 allows sufficient chain flexibility for nanocarrier self-assembly at room temperature via a wide range of methods^{7,9,10}.

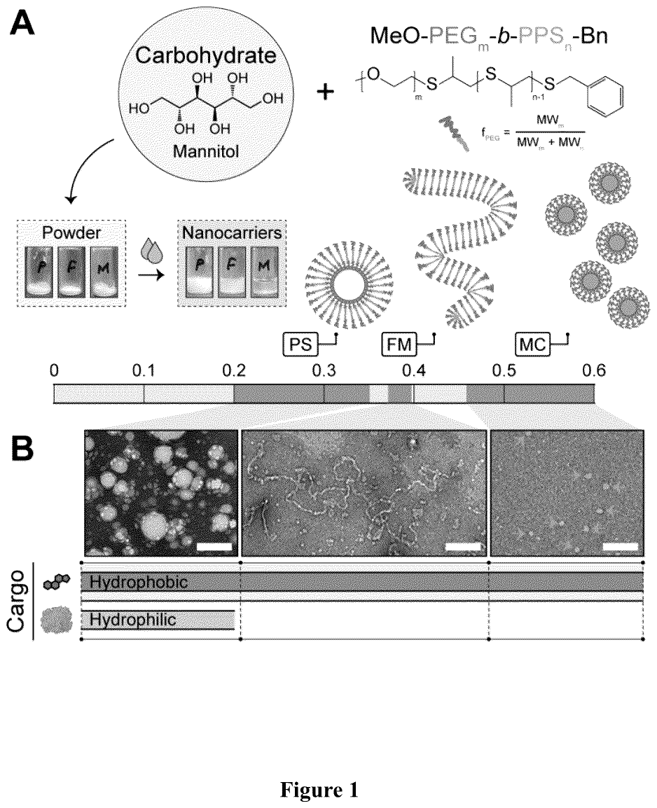

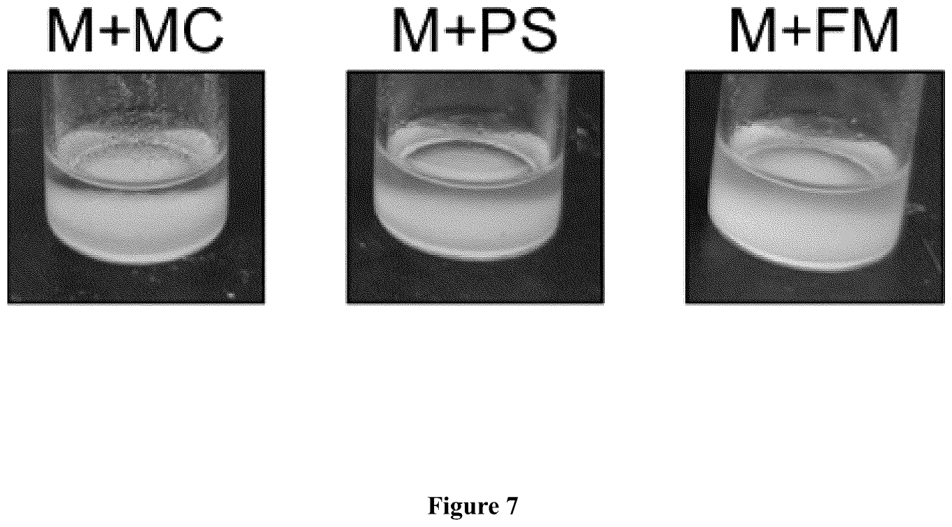

We hypothesized that suitable mixtures of polymer amphiphiles and carbohydrates could rapidly yield stable nanocarriers upon the addition of aqueous media. Formulations were developed consisting of a 1:3 ratio (polymer: carbohydrate) of PEG-b-PPS polymer and mannitol (FIG. 1A). Mannitol is a sugar alcohol and is commonly employed as an excipient in the pharmaceutical industry²⁷, including as a cryoprotectant for biomedical products^{28,29} Furthermore, Mannitol is widely used in the development of carbohydrate-based proliposome formulations¹¹. To examine whether MC, FM, or PS morphologies favourably self-assemble upon rehydration, we prepared powders with PEG-b-PPS diblock copolymers that differ in their hydrophilic weight fraction (FIG. 1A; FIG. 7; Table 1).

TABLE 1
PEG-b-PPS polymers used in this study

| Morphology | Polymer |
|---|---|
| Micelle (MC) | PEG₄₅-b-PPS₂₃ |
| polymer | |
| Filomicelle (FM) | PEG₄₅-b-PPS₄₄ |
| polymer | |
| Polymersome (PS) | PEG₂₃-b-PPS₂₈ |
| polymer | |

Morphological analysis by transmission electron microscopy (TEM) of negatively stained specimens demonstrates that spherical and cylindrical morphologies self-assemble successfully upon rehydration (FIG. 1B). The diameter of PEG-b-PPS PS exceeded that of the MC, as expected²³. FM had a cross-sectional diameter that was comparable to that of the MC, but a length exceeding 1 μm (FIG. 1B). This is in agreement with our past reports on these worm-like structures^{3,23}.

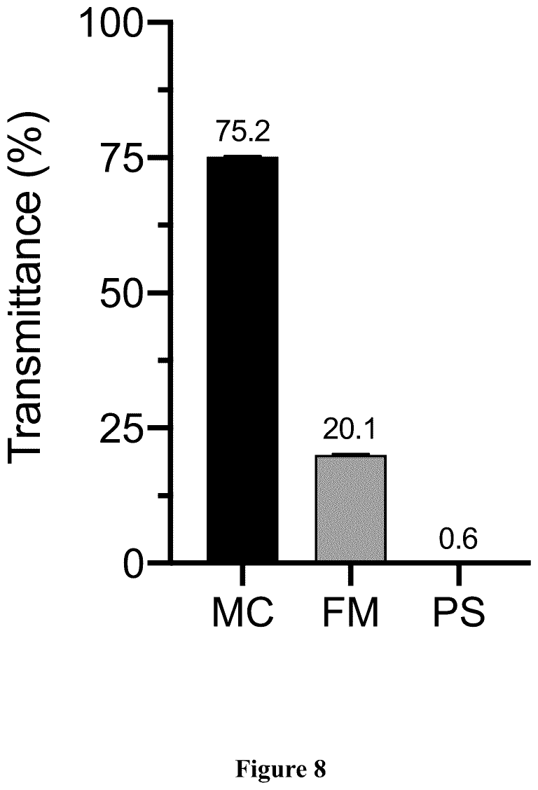

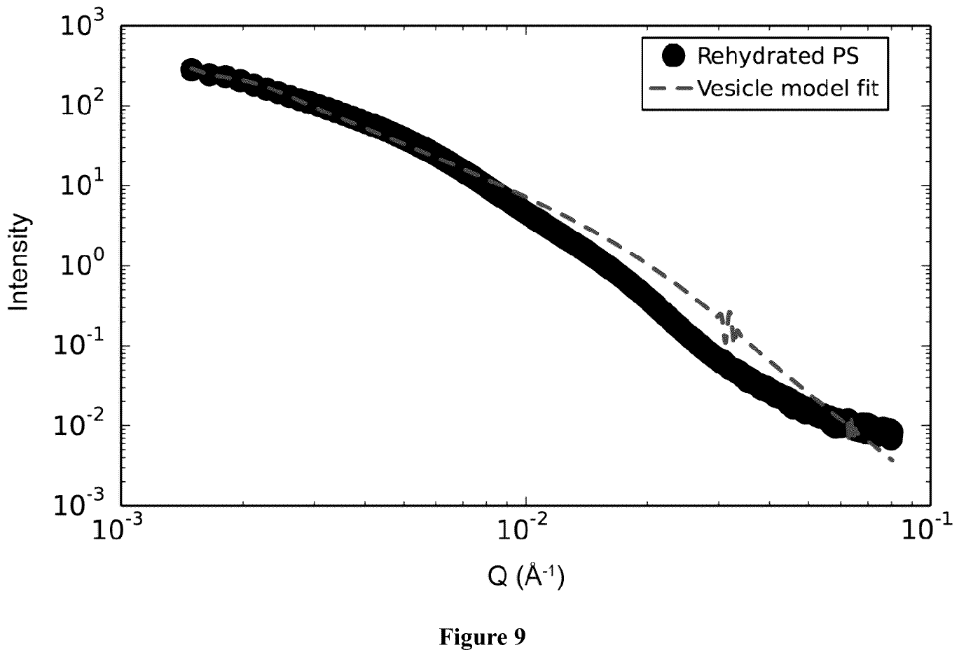

Differences in nanocarrier size are also readily observable through transmittance measurements (FIG. 8). As expected, MC formulations are transparent, FM are translucent, and the vesicular PS are more opaque (FIG. 8). Results for spherical morphologies were corroborated by dynamic light scattering (DLS) (Table 2). Rehydrated PEG-b-PPS MC and PS were monodisperse with an average diameter of 22.6 nm and 78.5 nm, respectively (Table 2). The vesicular morphology of PS was confirmed by small-angle x-ray scattering (SAXS) using synchrotron radiation (FIG. 9).

TABLE 2
Summary of rehydrated nanocarrier physicochemical
properties, cargo loading, and ligand display

| | | Efficiency | | | |
|---|---|---|---|---|---|
| Nanocarrier | Cargo or Ligand | (%)* | D (nm)^{†} | PDI^{†} | □(mV)^{††} |
| Micelles (MC) | None | N/A | 22.6 | 0.06 | −5.0 ± 0.9 |
| DiI MC | DiI | >95.0 | 26.5 | 0.07 | −2.2 ± 0.8 |
| MC + peptide | Targeting ligand | >95.0 | 26.7 | 0.08 | −0.3 ± 0.3 |
| Filomicelles (FM) | None | N/A | — | — | −7.5 ± 0.5 |
| DiI FM | DiI | >95.0 | — | — | −7.2 ± 0.2 |
| FM + peptide | Targeting ligand | >95.0 | — | — | −0.3 ± 0.2 |
| Polymersomes (PS) | None | N/A | 78.5 | 0.12 | −4.4 ± 0.1 |
| DiI PS | DiI | >95.0 | 75.0 | 0.12 | −1.1 ± 0.2 |
| PS + peptide | Targeting ligand | >95.0 | 72.3 | 0.16 | 9.8 ± 0.1 |
| AP PS | AP^{†††} | 16.9 ± 0.2 | 82.9 | 0.12 | −3.0 ± 0.6 |
| Dex-TMR PS | Dex-TMR^{†††} | 10.8 ± 0.5 | 73.9 | 0.09 | −0.5 ± 0.3 |

*Efficiency (%) = loading efficiency for cargo; display efficiency for targeting ligands.
^{†}Number-average diameter and polydispersity index (PDI) determined by DLS.
Note:
FM (cylinders) are excluded from DLS analysis (Stokes-Einstein equation assumes spheres).
^{††}Zeta potential is reported as mean ± s.d. (n = 3).
^{†††}Hydrophilic cargo (only applicable to the vesicular PS morphology)

We further assessed the storage properties of the powdered formulations and the rehydrated nanocarrier suspensions, where relevant. Nanocarriers formed by hydrating powders for 1 month or 6 months were monodisperse, with physical properties similar to those of nanocarriers formed from freshly prepared powders (Table 3). This is particularly important for micellar nanocarriers, which often have stability issues in storage in suspension form. The resulting powders avoid these issues since they exhibit high storage stability and can be rehydrated on demand to prepare formulations with minimal effort.

TABLE 3
Rehydrated nanocarriers prepared from stored powders

| | | | Avg. Diameter | |
|---|---|---|---|---|
| Nanocarrier | Storage duration | | (nm)^{†} | PDI^{†} |
| MC | 1 | month | 29.4 | 0.08 |
| MC | 6 | months | 26.3 | 0.12 |
| PS | 1 | month | 96.1 | 0.11 |
| PS | 6 | months | 64.8 | 0.18 |

^{†}Number-average diameter and polydispersity index (PDI) determined by dynamic light scattering (DLS).

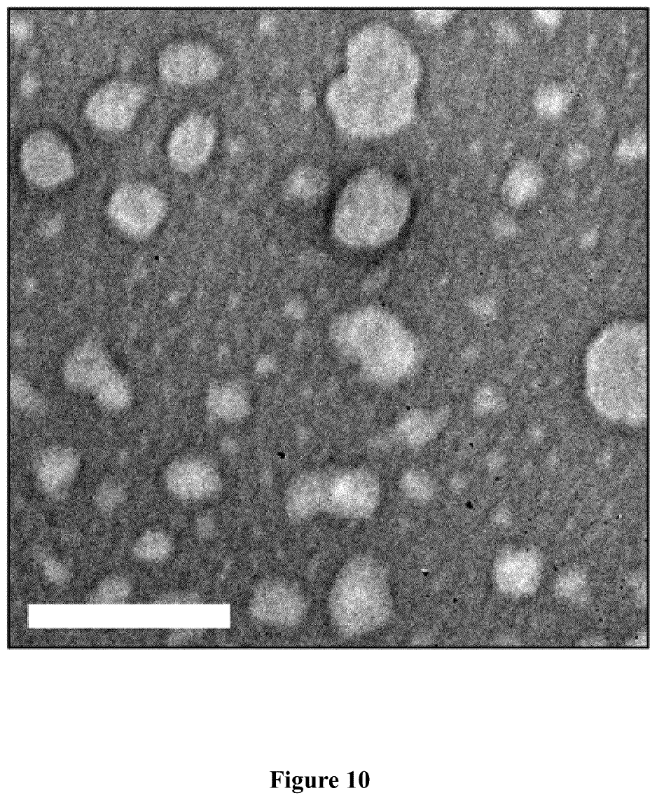

Due to their higher stability compared to MC³⁰, vesicular nanocarriers such as PS are more frequently stored in suspension form for longer periods of time. In our studies, rehydrated suspensions of PS stored at room temperature for 8 months remained stable. The stored PS formulations were monodisperse with an average diameter of 68.9 nm (Table 4). PS were still readily observable after storage for 1 year at room temperature (FIG. 10).

TABLE 4
Storage stability of rehydrated polymersome suspensions

| | | Avg. Diameter | |
|---|---|---|---|
| Nanocarrier | Storage duration | (nm)^{†} | PDI^{†} |
| PS | Before storage | 77.5 | 0.15 |
| PS | 8 months | 68.9 | 0.14 |

^{†}Number-average diameter and polydispersity index (PDI) determined by dynamic light scattering (DLS).

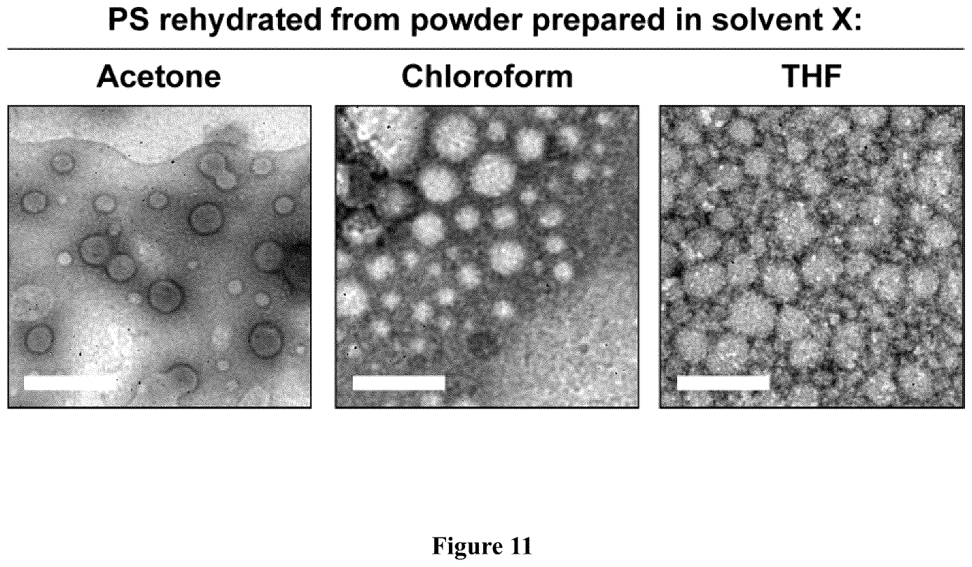

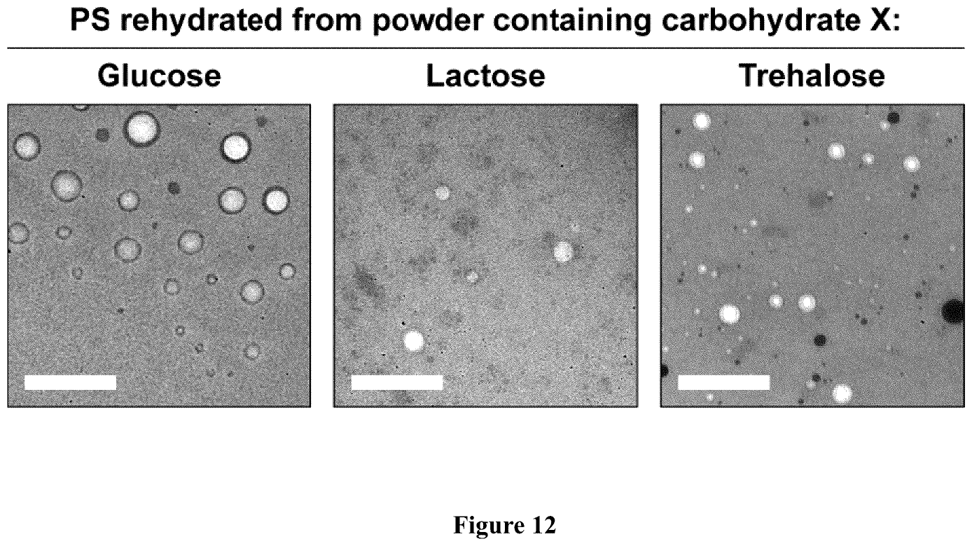

For completeness, we note that nanocarrier self-assembly upon rehydration was also permitted using different volatile organic solvents (FIG. 11) and carbohydrates (FIG. 12). Nanocarriers formed from powders that were prepared with alternative solvents (Table 5) and carbohydrates (Table 6) were monodisperse with an average diameter consistent with nanocarriers prepared from powders formed with mannitol and dichloromethane (Table 2).

TABLE 5
Rehydrated nanocarriers from mannitol + polymer
powders prepared using alternative organic solvents

| | | Avg. Diameter | |
|---|---|---|---|
| Nanocarrier | Solvent | (nm)^{†} | PDI^{†} |
| PS | Acetone | 64.6 | 0.30 |
| PS | Chloroform | 80.5 | 0.17 |
| PS | THF | 85.7 | 0.18 |

^{†}Number-average diameter and polydispersity index (PDI) determined by dynamic light scattering (DLS).

TABLE 6
Rehydrated nanocarriers from powders prepared
in THF with alternative carbohydrates

| | | Avg. Diameter | |
|---|---|---|---|
| Nanocarrier | Carbohydrate | (nm)^{†} | PDI^{†} |
| PS | Glucose | 69.5 | 0.19 |
| PS | Lactose | 54.1 | 0.21 |
| PS | Trehalose | 78.1 | 0.14 |

^{†}Number-average diameter and polydispersity index (PDI) determined by dynamic light scattering (DLS).

### Morphological and Crystallographic Characterization of Polymer-Carbohydrate Powders

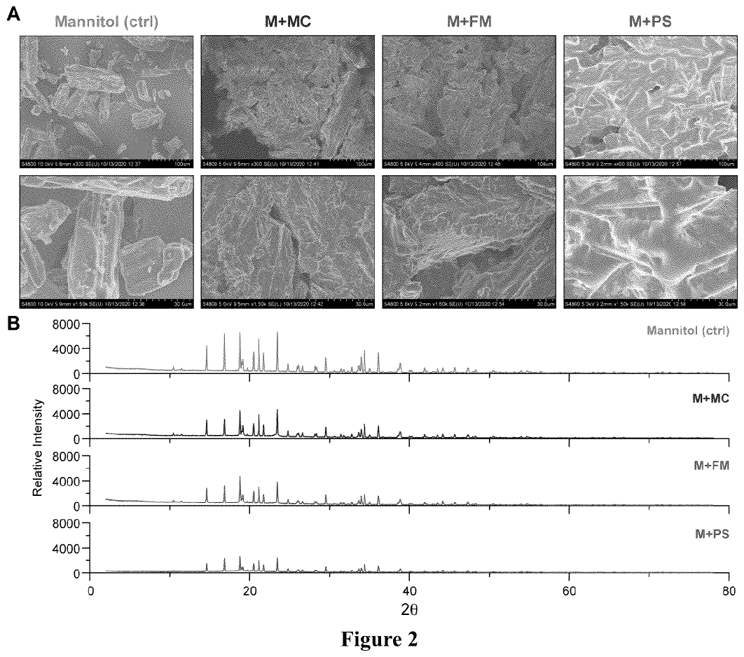

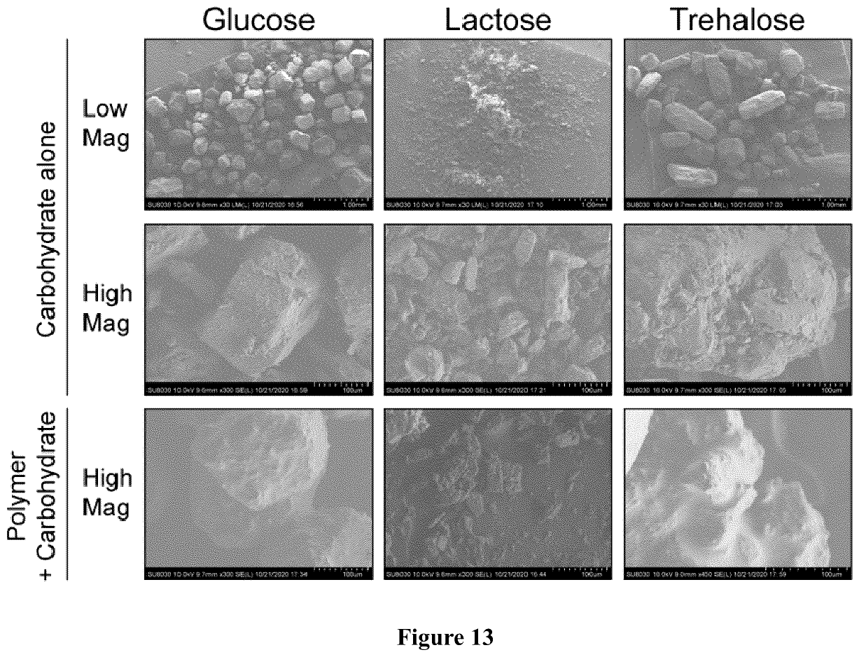

Employed as an excipient, the crystallinity of mannitol, as well as other carbohydrates, is known to influence its utility as a stabilizing agent for retaining the activity of lyophilized enzymes³¹⁻³³, and in promoting the self-assembly of pro-liposomes¹¹. To examine the structural properties and crystallinity of the polymer-carbohydrate powders in greater detail, we characterized each formulation using scanning electron microscopy (SEM) and powder x-ray diffraction (XRD) (FIG. 2). In all powder formulations, PEG-b-PPS polymers coated the crystalline mannitol carbohydrate. This is observable by SEM as a smooth layer that renders the uncoated carbohydrate to have a more amorphous and rough appearance (FIG. 2A). Similar observations were made for powders prepared with alternative carbohydrates, where a smooth polymer coating is visible by SEM (FIG. 13).

XRD analysis further confirms our interpretations of SEM micrographs (FIG. 2B). Diffuse peaks of lower intensity are observed in polymer-coated mannitol samples compared to mannitol control (FIG. 2B). This peak broadening is observed due to the coating of disordered polymer amphiphiles onto the surfaces of ordered mannitol crystals, which disrupts x-ray diffraction. These analyses demonstrate the powder formulations consist of self-assembling polymer amphiphiles that integrate stably with the carbohydrate additives.

### Rehydrated Nanocarriers Retain Diverse Hydrophobic Payloads, are Non-Toxic, and are Differentially Endocytosed by Immune Cells.

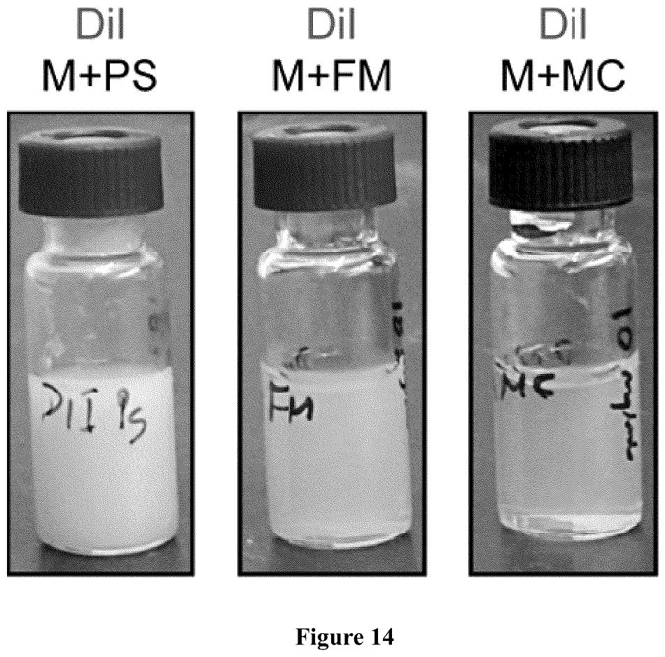

Nanocarriers hold broad utility, including their usage in drug delivery and imaging for transporting payloads having low water solubility to specific tissues and cell types. We therefore prepared powder formulations containing hydrophobic cargos and assessed loading efficiency upon rehydration. All nanocarriers loaded DiI hydrophobic dye with >95% efficiency (Table 2). Representative images of rehydrated DiI-loaded nanocarrier formulations are presented in FIG. 14. The high loading efficiency of DiI is consistent with our previously reported work for PEG-b-PPS nanocarriers loading lipophilic tracers, prepared via thin-film rehydration^{3,17}, flash nanoprecipitation^{7,10,18}, and cosolvent evaporation^{2,34}. More modest loading efficiencies were found for curcumin (Table 7), which is explainable by its relatively lower hydrophobicity than DiI. Physicochemical analysis by DLS demonstrates that cargo loading did not produce substantial changes to nanocarrier size, and the resulting nanocarrier suspensions were monodisperse (Table 2). Electrophoretic light scattering (ELS) analysis further demonstrates zeta potential was not altered by cargo loading (Table 2).

TABLE 7
Physicochemical properties and hydrophobic loading of rehydrated
nanocarriers after polymer-carbohydrate rehydration

| | | Loading | Avg. | | Zeta |
|---|---|---|---|---|---|
| | Hydrophobic | Efficiency | Diameter | | Potential |
| Nanocarrier | Cargo | (%) | (nm)^{†} | PDI^{†} | (mV)^{††} |
| C-MC | Curcumin | 36.9 ± 2.5 | 24.3 | 0.08 | −5.4 ± 0.8 |
| C-PS | Curcumin | 27.6 ± 1.6 | 68.9 | 0.30 | −2.3 ± 0.4 |
| Dex-TMR, | Dex-TMR | 16.3 ± 1.1 | 73.9 | 0.09 | −1.8 ± 0.1 |
| Curcumin PS | Curcumin | 22.9 ± 0.4 | | | |
| (dual loaded) | | | | | |

^{†}Number-average diameter and polydispersity index (PDI) determined by dynamic light scattering (DLS).
^{††}Zeta potential is reported as mean ± s.d.

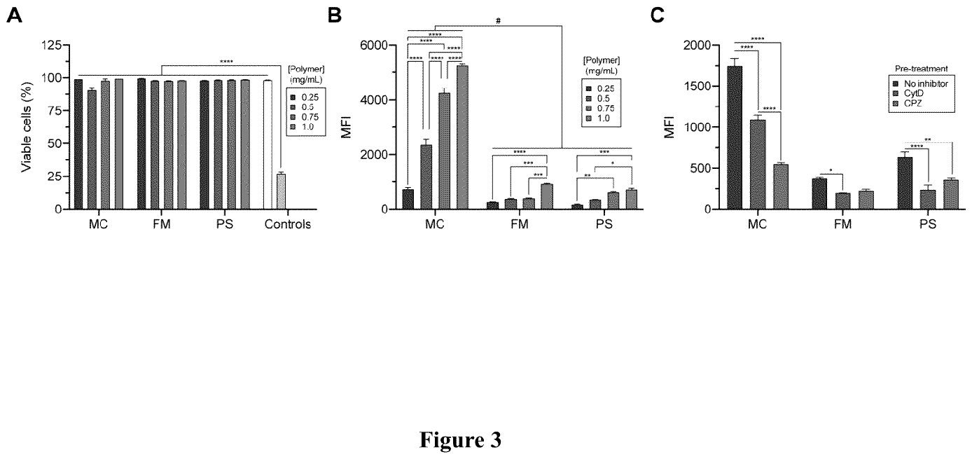

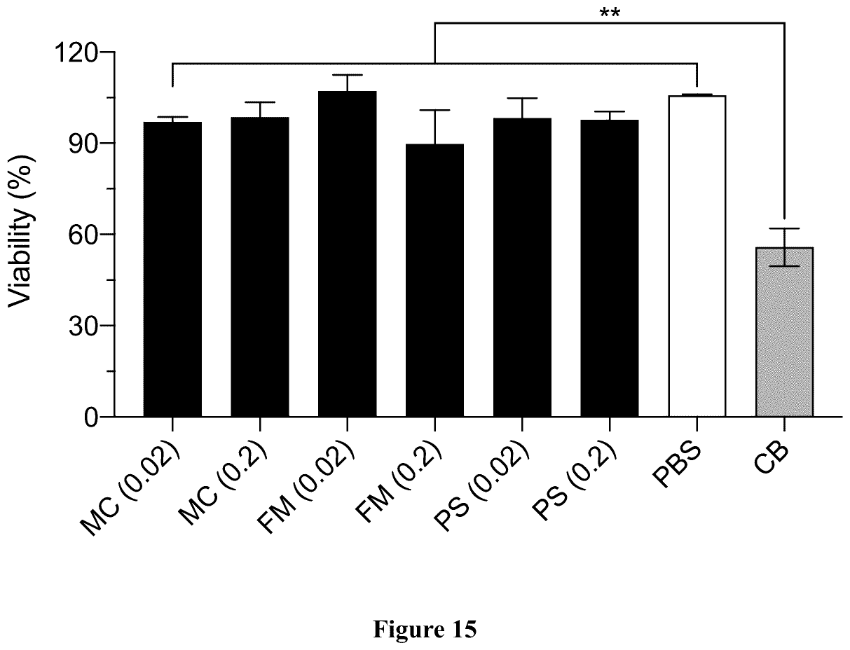

While cytotoxic nanocarrier systems are useful for cancer therapy, it is generally favorable for the nanocarrier to be biologically inert and non-toxic at relevant concentrations. Greater than 85% viability was observed for macrophages cultured in the presence of rehydrated nanocarriers dosed by polymer concentration in the range of 0.25-1.0 mg/mL for 24 h (FIG. 3A). With exception to the 0.5 mg/mL MC treatment group, all viabilities exceeded 90% (FIG. 3A). For comparison, brief cellular exposure to 70% ethanol (toxicity control), decreased cell viability to below 30% (FIG. 3A). Comparing cell viability in the presence of nanocarriers (>85% mean viability in all cases) suggests all rehydrated nanocarriers were non-toxic at the high concentrations administered. As an orthogonal assessment of toxicity, an MTT assay further demonstrate the nanocarriers to be non-toxic over 24 h. In these studies, the viability of cells treated with rehydrated nanocarriers was significantly higher than that of cells treated with water-soluble carboplatin, a cytotoxic anti-cancer agent (FIG. 15).

We next quantified differential cellular uptake by assessing the difference in the median fluorescence intensity (MFI) (FIG. 3B). For each rehydrated DiI-loaded nanocarrier, the MFI generally increased in a concentration-dependent fashion (FIG. 3B). Furthermore, larger nanostructures, PS and FM, were taken up by macrophages at a lower rate than the rehydrated MC at similar concentrations, as expected due to differences in diffuse rate and nanoparticle count (FIG. 3B). Cellular uptake studies performed after cellular pre-treatment confirmed our findings of morphology-dependent internalization rates. Both cytochalasin D (CytD) 35, a general phagocytosis inhibitor, and chlorpromazine (CPZ) 36, an inhibitor of clathrin-mediated endocytosis, decreased uptake of all structures (FIG. 3C). Collectively, these viability and cellular uptake studies demonstrate the rehydrated polymeric nanocarriers are non-toxic to immune cells and are differentially internalized by cells at a rate that is size- and morphology-dependent. The resulting nanocarriers therefore fulfil basic requirements for the intracellular delivery of drugs, vaccines, and tracers/diagnostics.

### Preparation of Rehydratable, Morphologically Diverse Targeted Drug Delivery Vehicles

Targeted drug delivery vehicles consist of drug-loaded nanocarriers displaying a ligand that binds to a unique molecular feature that is enriched on the surface of one or more cell type(s) of interest. The goal of these vehicles is to increase the drug concentration at the site of action to improve efficacy, while minimizing side effects that are associated with off-target drug uptake. Lipid-anchored targeting ligands offer a modular approach for functionalizing amphiphilic nanocarriers for targeted drug delivery applications without the need for covalent modification of the polymer. Powdered formulations that consist of a self-assembling polymer amphiphile, lipid-anchored targeting ligand, and carbohydrate additive would offer a rapid and customizable platform for facile on-demand preparation of targeted drug delivery vehicles.

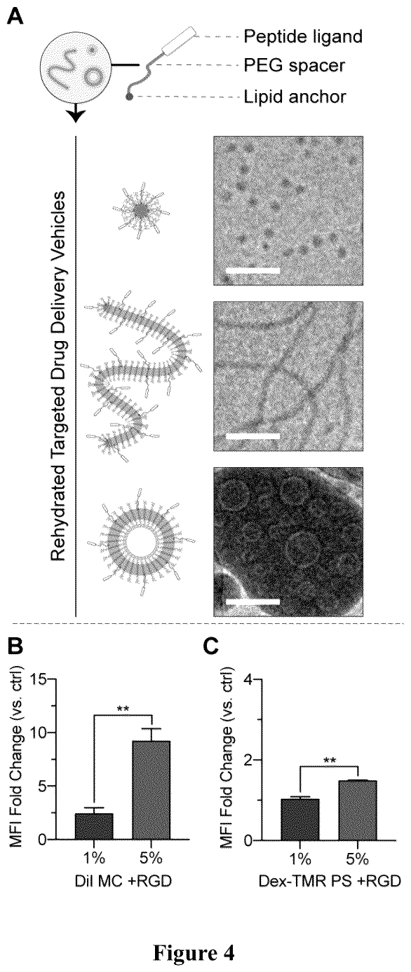

We prepared powders consisting of PEG-b-PPS polymer with weight fractions that self-assemble into MC, FM, or PS morphologies, mannitol, and a model lipid-anchored targeting peptide of the form palmitoleic acid-PEG₆-peptide (FIG. 4A). Powders were prepared to include a linear peptide at a 5% molar ratio (peptide: polymer). Our past targeting studies demonstrate this 5% molar ratio yields an optimal ligand-mediated increase in uptake by the target cell type, whereas embedding ligand at higher molar ratios does not produce substantial increases in targeting enhancements^{34,37}. After rehydration, the nanocarriers successfully embedded the lipid-anchored targeting peptides at over >95% efficiency (Table 2).

Cryo-TEM was performed to examine the morphology of the targeted drug delivery vehicles formed after rehydration (FIG. 4A). For Cryo-TEM performed on PEG-b-PPS nanocarriers, the dense hydrophobic PPS core is the source of contrast, whereas the PEG corona is not readily visible due to its insufficient contrast. As demonstrated by representative Cryo-TEM micrographs, the expected morphologies were retained in the presence of peptide embedding (FIG. 4A). Aberrations in the hydrophobic PPS core due to the lipid anchor were not observed (FIG. 4A), and the morphologies did not deviate from the nanocarriers prepared in the absence of targeting peptide (FIG. 1B). DLS and ELS demonstrate the spherical structures to be monodisperse with a diameter comparable to that of nanocarriers prepared in the absence of lipid-anchored targeting ligand (Table 2).

We examined targeting functionality in vitro. The model cyclic RGD peptide was used for these studies, which exhibits greater stability/lower degradation than its linear counterparts^{38,39} and have been used in a large number of targeting applications⁴⁰⁻⁴². RGD peptides bind to beta integrin receptors⁴³⁻⁴⁶, which are present on the surface of a variety of endothelial cell types⁴⁷, including human umbilical vein endothelial cells (HUVECs)⁴⁸ For this analysis, micelle powders were prepared with DiI as a model hydrophobic cargo whereas PS powders were prepared with 70 kDa Dex-TMR as a model hydrophilic cargo. Lipid-anchored cyclic RGD peptides of the form, palmitoleic acid-PEG₆-cyclic RGD, were added at 1% or 5% molar ratios (peptide: polymer) and formed stable nanostructures (Table 8). HUVECs were treated with rehydrated formulations and peptide-mediated enhancements in cellular uptake were quantified versus untargeted control (FIG. 4B, C). For both cases, the display of cyclic RGD at the nanocarrier surface-enhanced uptake and the magnitude of the increase was significantly greater for nanocarriers displaying the peptide at a 5% molar ratio (FIG. 4B, C).

TABLE 8
Physicochemical properties of nanocarriers
displaying cyclic RGD targeting peptides

| | Avg. Diameter | | Zeta Potential |
|---|---|---|---|
| Formulation | (nm)^{†} | PDI^{†} | (mV)^{††} |
| DiI MC | 27.4 | 0.09 | −1.1 ± 0.1 |
| DiI MC + 1% | 22.1 | 0.20 | −1.3 ± 0.4 |
| RGD | | | |
| DiI MC + 5% | 27.7 | 0.07 | 0.1 ± 0.9 |
| RGD | | | |
| Dex-TMR PS | 111.8 | 0.16 | −0.6 ± 0.2 |
| Dex-TMR | 87.5 | 0.14 | −0.4 ± 0.2 |
| PS + 1% RGD | | | |
| Dex-TMR | 84.2 | 0.17 | 4.1 ± 0.3 |
| PS + 5% RGD | | | |

^{†}Number-average diameter and polydispersity index (PDI) determined by dynamic light scattering (DLS).
^{††}Zeta potential is reported as mean ± s.d.

These results demonstrate the powder-based formulation strategy established herein can form morphologically diverse polymeric targeted drug delivery vehicles. The surface display of the model cyclic RGD peptide enhanced the micellar delivery of a hydrophobic tracer cargo and PS delivery of a biological tracer. The powdered form bypasses ligand stability issues common to stored liquid suspensions, which could otherwise diminish targeting performance with time. The modularity of these powders and the diversity of the rehydrated targeted drug delivery vehicles enable their convenient preparation for a broad range of applications where precise control over nanocarrier-cellular interactions is required.

### Rehydrated Vesicles Load Diverse Hydrophilic Cargo and Preserve Enzyme Bioactivity

We next sought to examine the properties unique to the vesicular nanocarriers, such as facile encapsulation of water-soluble payloads. PS are capable of loading biologics, such as carbohydrates, nucleic acid, and protein cargo into their aqueous lumen. Alkaline phosphatase (AP) and 70 kDa dextran-tetramethylrhodamine (Dex-TMR) were chosen as two model hydrophilic cargo. Alkaline phosphatase is an enzyme that is capable of non-specific dephosphorylation, which we selected to assess retention of enzyme activity following our powder nanocarrier loading protocol⁴⁹. Dex-TMR is a high molecular weight polysaccharide conjugated to TMR fluorophore, and is commonly employed as a hydrophilic tracer¹⁸.

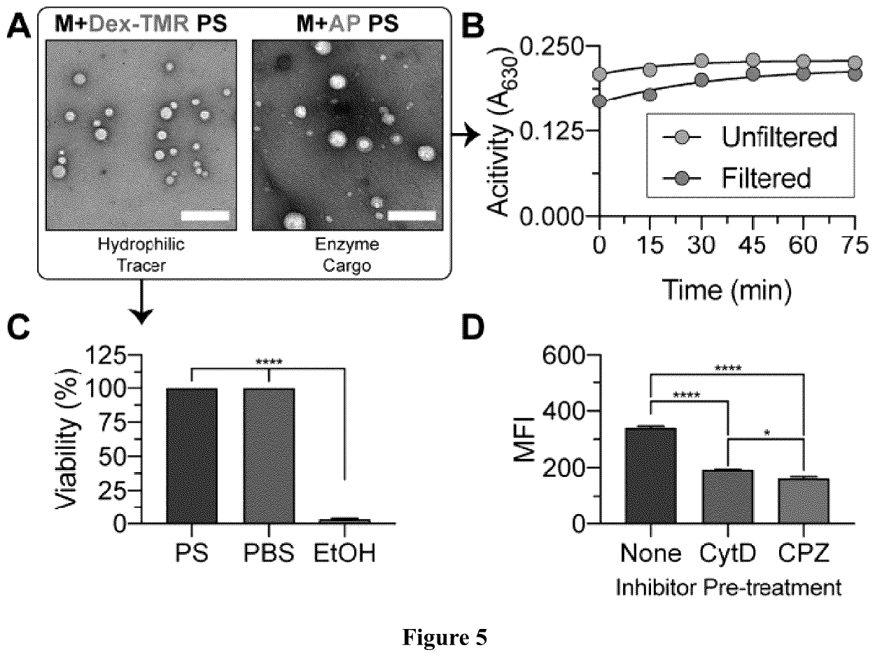

The enzyme cargo was prepared in aqueous media, which was then used to rehydrate powder consisting of PEG-b-PPS PS polymer and mannitol carbohydrate. Following rehydration, AP and Dex-TMR loaded into PS at 16.9±0.2% and 10.8±0.5%, respectively (Table 2). These results are consistent with the observation that higher molecular weight hydrophilic cargo load into PS at greater efficiency (AP molecular weight >100 kDa). Furthermore, rehydrated PS formations are also capable of dual loading hydrophobic and hydrophilic cargo. The rehydrated PS dual loaded Dex-TMR (hydrophilic cargo) and curcumin (hydrophobic cargo) with a loading efficiency of 16.3±1.1% and 22.9±0.4%, respectively (Table 7). The resulting PS bearing biological cargo are monodisperse, with physicochemical characteristics similar to that of PS prepared without cargo or with hydrophobic cargo (Table 2). TEM demonstrates the PS nanocarriers have the expected spherical morphology (FIG. 5A) and do not deviate substantially from unloaded structures (FIG. 1B).

AP-loaded PS were either left unpurified (a mixture of free, unloaded AP and AP loaded into PS) or were purified by size exclusion chromatography (SEC) using a Sepharose 6B column. The latter condition permits the examination of substrate turnover by the encapsulated enzymes, without confounding effects from product formation mediated by the diffuse enzyme that was not loaded into nanostructures. Filtered or unfiltered AP-loaded PS aliquots were treated with 1% triton to break the PS structure, and 5-bromo-4-chloro-3-indolyl phosphate (BCIP)/nitro blue tetrazolium (NBT) substrate was administered. AP retained bioactivity following encapsulation into PS (FIG. 5B). PS loaded with Dex-TMR hydrophilic tracer are non-toxic to macrophages (FIG. 5C) and are readily endocytosed by macrophages in vitro (FIG. 5D).

Our analyses demonstrate the PS powders permit the encapsulation of hydrophilic cargo with efficiency on par with most current hydrophilic encapsulation methods. However, we note that this loading efficiency is lower than that achieved by flash nanoprecipitation^{7,18} The results presented herein demonstrate that encapsulated enzymes retain bioactivity. Furthermore, PS bearing hydrophilic tracers are non-toxic and their cellular internalization is readily detectable.

### Self-Assembly of Polymeric Nanostructures Upon Rehydration of Commercially Available Polymers

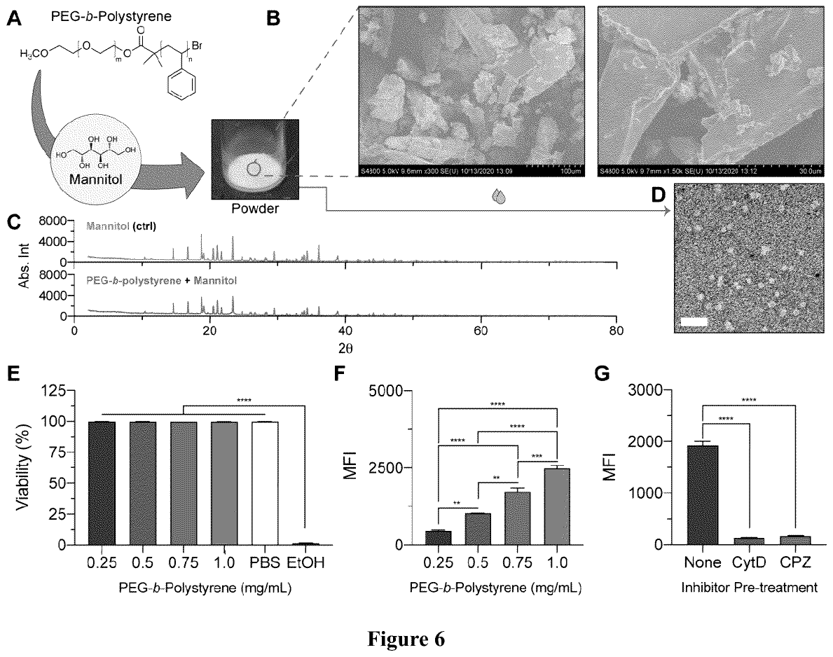

We found the utility of the developed formulation strategy extends beyond the PEG-b-PPS platform and is generally applicable to self-assembling polymer amphiphiles having low glass transition temperature. This includes polymer systems that are commercially available, such as PEG-b-polystyrene polymer that forms micelles based on its f_{PEG} ratio (FIG. 6A; Table 9). As observed through SEM, PEG-b-polystyrene polymer coats mannitol carbohydrate to produce a more amorphous appearance of the formed powder (FIG. 6B). Further support for successful coating is provided by the XRD profile of the polymer-carbohydrate powder, where peak broadening is observed together with decreases in peak intensity (FIG. 6C).

TABLE 9
Physicochemical characterization of rehydrated nanocarriers self-
assembled from powders containing commercially available polymers

| | Loading | Avg. | | Zeta |
|---|---|---|---|---|
| | Efficiency | Diameter | | Potential |
| Nanocarrier | (%) | (nm)^{†} | PDI^{†} | (mV)^{††} |
| PEG-b-polystyrene | N/A | 39.8 | 0.06 | −8.5 ± 0.8 |
| PEG-b-polycaprolactone | N/A | 33.5 | 0.18 | −3.2 ± 0.1 |

MC self-assembled after rehydrating PEG-b-polystyrene powders with aqueous media (FIG. 6D). These formulations were monodisperse, with an average diameter of 39.8 nm and zeta potential of −8.5±0.8 (Table 9). PEG-b-polystyrene nanocarriers encapsulated hydrophobic small molecules at high efficiency (Table 10). DiI-loaded PEG-b-polystyrene nanocarriers were non-toxic to macrophages in vitro (FIG. 6E). Macrophages internalized these nanocarriers in a manner that was concentration-dependent (FIG. 6F). The cellular uptake of these nanocarriers is mostly abolished by pre-treating macrophages with endocytosis inhibitors prior to nanocarrier administration (FIG. 6G).

TABLE 10
Commercial nanocarrier loading

| | | | Loading |
|---|---|---|---|
| | | | Efficiency |
| Nanocarrier | Morphology | Cargo | (%) |
| PEG-b-polystyrene | Micelle | DiI (hydrophobic) | 86.5 ± 4.4 |
| PEG-b-polycaprolactone | Polymersome | DiI (hydrophobic) | 45.5 ± 2.1 |
| | | Dex-TMR | 9.6 ± 0.5 |
| | | (hydrophilic) | |

Powders were also prepared with PEG-b-polycaprolactone to examine the ability to produce rehydratable vesicular nanocarriers from a commercially available polymer. PEG-b-polycaprolactone is expected to form PS based on its f_{PEG} ratio. After rehydration, PEG-b-polycaprolactone self-assembly required a heating step at 60° C. for 30 min after rehydration with aqueous media to allow the formation of monodisperse vesicles (Table 9). However, this short timeline is still a convenient approach to fabricate PEG-b-polycaprolactone vesicles as compared to the thin-film hydration method that traditionally requires an overnight incubation at 60° C. to self-assemble monodisperse vesicular nanostructures⁵⁰. Furthermore, PEG-b-polycaprolactone powders successfully permit the encapsulation of both hydrophilic and hydrophobic cargo without comprising the structural integrity of the nanocarrier, which is consistent with successful vesicle formation (Table 10).

### Conclusions

We developed a carbohydrate-based powdered formulation strategy that permits nanocarrier self-assembly from polymer amphiphiles after the addition of water or saline. The formulated powders are robust, accommodating a variety of carbohydrates and organic solvents. We further demonstrate this formulation strategy is extendable to unique polymer systems, such as PEG-b-PPS, as well as polymer amphiphiles that are commercially available. Importantly, powders incorporating polymer amphiphiles of distinct hydrophilic weight fractions self-assembled into the expected morphologies upon rehydration, as demonstrated by the successful formation of MC, FM, and PS. Rehydrated micellar and vesicular nanocarriers load hydrophobic payloads with high efficiency. Rehydrated vesicular PS encapsulated diverse hydrophilic payloads without exposure to organic solvents and retained the bioactivity of enzyme cargo. The rehydratable PS were also capable of dually loading hydrophobic and hydrophilic cargoes. In all cases, the rehydrated nanocarriers were non-toxic, and were internalized by cells at relative rates that is consistent with the combination of nanocarrier size and shape. All nanocarrier morphologies could display modular lipid-anchored targeting ligands. Our studies with the model cyclic RGD peptide demonstrated the successful enhancement of MC, FM, and PS uptake by HUVECs.

Finally, the powders remain stable after long-term storage. Powders stored for a period of 6 months successfully form monodisperse nanocarriers following rehydration. This feature bypasses numerous issues associated with the storage of nanocarrier suspensions, which is an issue that gained worldwide attention during the development of vaccines against COVID-19⁵¹. The powder formulations developed herein solve numerous issues surrounding the preparation and stability of polymeric nanocarriers of diverse morphology. These technologies have potential utility in a variety of drug delivery and commercial applications.

### Materials & Methods

Chemicals. Unless stated otherwise, all chemical materials and reagents were purchased from MilliporeSigma (St. Louis, MO).

PEG-b-PPS synthesis. PEG-b-PPS polymer was synthesized using established procedures described extensively elsewhere^{3,17,18,23,24}. Briefly, sodium methoxide was used to deprotect PEG thioacetate. This deprotected PEG thioacetate was then used to initiate the polymerization of propylene sulfide through an anionic ring-opening polymerization reaction. PEG₇₅₀ was used as the hydrophilic block of polymersome polymer, whereas PEG₂ₖ was used as the hydrophilic block for micelle and filomicelle polymers. To promote the self-assembly into micelle, filomicelle, or polymersome morphologies, polymers were synthesized with a specific hydrophilic weight fraction (f_{PEG}), as determined by the relative lengths of PEG starting material and the polymerized PPS chain. A summary of each polymer type used in this study is presented in Table 11.

TABLE 11
PEG-b-PPS polymers used in this study

| Morphology | Polymer |
|---|---|
| Micelle (MC) polymer | PEG₄₅-b-PPS₂₃ |
| Filomicelle (FM) polymer | PEG₄₅-b-PPS₄₄ |
| Polymersome (PS) polymer | PEG₂₃-b-PPS₂₈ |

Peptide synthesis. Fmoc-N-amido-dPEG₆-acid was purchased from Quanta Biodesign for use as a PEG spacer. A peptide construct of the form palmitoleic acid-PEG₆-WHWLPNLRHYAS (SEQ ID NO:1) was synthesized by solid phase peptide synthesis. This peptide is specific for FLT4 receptor found on cells of lymphatic origin, including Schlemm's Canal cells in the eye and lymphatic endothelial cells found in lymphatic vessels. The lipid anchored peptide product was >95% pure, as assessed by LC-MS.

Preparation of synthetic polymer-carbohydrate powders. Powders capable of promoting stable nanocarrier self-assembly upon rehydration were prepared using a slurry method. First, PEG-b-PPS polymer (10 mg) was dissolved in a volatile organic solvent, dichloromethane (DCM). Carbohydrate (30 mg; mannitol, trehalose, or glucose) was weighed separately and transferred into a 2 ml glass vial. The organic phase containing the polymer was subsequently added to the carbohydrate powder and mixed using a pipette to form a slurry. Where specified for hydrophobic loading and targeting peptide-display studies, hydrophobic drug cargo or targeting peptide was dissolved in an organic solvent and was subsequently added to this mixture prior to solvent removal. The solvent was evaporated in a vacuum desiccator overnight. Powders containing different carbohydrates (lactose, trehalose, glucose) were also prepared in a similar way as described above using DCM as the organic solvent. Where specified, different volatile organic solvents (acetone, tetrahydrofuran, and chloroform) were also utilized to form the powder formulations using mannitol as the carbohydrate.

Preparation of commercially available polymer amphiphile powders. PEG-b-polystyrene and PEG-b-poly(e-caprolactone) were obtained from Polymer Source, Inc. 10 mg of PEG-b-polystyrene or PEG-b-poly(e-caprolactone) were weighed separately in a vial and dissolved in DCM. The polymer solution was then added to the 2 ml vial containing 30 mg of Mannitol and mixed using a pipette to form a slurry. The organic solvent was subsequently removed overnight in the vacuum desiccator. For loading studies, the hydrophobic cargo was mixed in the polymer solution before adding to the carbohydrate.

Powder X-ray diffraction (PXRD). PXRD data were collected at room temperature on an STOE-STADI-P powder diffractometer equipped with an asymmetric curved Germanium monochromator (CuKα1 radiation, λ=1.54056 Å) and one-dimensional silicon strip detector (MYTHEN2 1K from DECTRIS). The line focused Cu X-ray tube was operated at 40 kV and 40 mA. The powder was packed in 3 mm metallic mask and sandwiched between two polyimide layers of tape. Intensity data from 2 to 78 degrees two theta were collected over a period of 30 mins. Instrument was calibrated against a NIST Silicon standard (640d) prior the measurement.

Preparation of rehydrated nanocarriers. Each vial of powder (40 mg) containing polymer-carbohydrate mixture were rehydrated with 0.5-1.0 mL of water or PBS (as specified). Samples were briefly vortexed (<1 min) prior to characterization and/or usage in studies. For PEG-b-poly(e-caprolactone) powders, after rehydration with aqueous media, samples were placed in 60° C. water bath for 30 min for the formation of monodispersed nanocarriers. For loading of hydrophilic molecules into nanocarriers, hydrophilic molecules were dissolved in water or PBS prior to rehydration.

Transmission electron microscopy (TEM) of negatively stained nanostructures. Nanocarrier samples were negative stained with uranyl formate. Briefly, a 1.0% uranyl formate (UF) negative stain was prepared in ultrapure water and was adjusted to pH 4.5 via the addition of 10 N KOH (2 μl of 10 N KOH/1 mL of UF). The prepared uranyl formate stain was filtered using a 0.45 μm nylon filter prior to use. Carbon-coated copper grids (400-mesh) were glow discharged (25 W, 10 s) and 3 μl of the nanoparticle sample was applied. Samples were blotted with Whatman filter paper, then were passed through two 30 μl volumes of water, followed by two 30 μl volumes of 1.0% UF. This procedure leaves ~0.5 μl stain on the grid, with an activity of 2.55×10⁻⁵ μCi/grid. Transmission electron microscopy was performed using a JOEL 1400 Transmission Electron Microscope operating at 120 kV. Images were acquired at 30,000× unless otherwise indicated.

Cryogenic transmission electron microscopy (Cryo-TEM). Lacey carbon Cu grids (200 mesh) were glow discharged using a Pelco easiGlow glow discharger (Ted Pella). The glow discharging procedure used an atmosphere plasma generated at 15 mA for 15 s with a 0.24 mbar pressure. A 4 μl volume was applied to the grid and was blotted for 5 s with a blot offset of +0.5 mm. Immediately after blotting, the grid was plunged into liquid ethane within a FEI Vitrobot Mark III plunge freezing instrument (Thermo Fisher Scientific). Grids were transferred to liquid nitrogen for storage. A Gatan Cryo Transfer Holder model 626.6 (Gatan) was used to keep plunge-frozen grids vitreous at −180° C., while viewing on a JOEL JEM1230 LaB6 emission TEM (JOEL USA) at 100 keV. Micrographs were acquired using a Gatan Orius SC1000 CCD camera Model 831 (Gatan).

Small-angle x-ray scattering (SAXS). The morphology of rehydrated polymersomes was characterized by SAXS performed using synchrotron radiation at the DuPont-Northwestern-Dow Collaborative Access Team (DND-CAT) beamline at the Advanced Photon Source (APS) maintain at Argonne National Laboratory (Argonne, IL, USA). A sample-to-detector distance of approximately 7.5 m was used together with 10 keV (2=1.24 Å) collimated x-rays and a 3 s exposure time. Silver behenate diffraction patterns were used to calibrate the q-range. Data was analyzed in the q-range of 0.001-0.5 Å. PRIMUS 2.8.3. was used for data reduction, and SasView 5.0 was used for model fitting. A spherical vesicle model was fit to the data using a chi square (X²) minimization procedure, and modeling was performed following established procedures¹⁸.

Quantification of peptide loading efficiency. A mass of peptide construct corresponding to a 5% molar ratio (peptide: polymer) was used to prepare powders consisting of polymer, carbohydrate, and lipid-anchored peptide ligand. Tryptophan fluorescence was used to measure the amount of peptide embedded in nanocarriers after purification using an LH-20 column. Tryptophan fluorescence was measured using a Shimadzu RF-6000 Fluorescence Spectrofluorometer (λ_{Ex}=270 nm, λ_{Em}=350 nm). Samples were prepared in a quartz cuvette (1 mm path length; Hellma USA, Inc.). Peptide measurements were calibrated against a peptide concentration series (0, 0.06125, 0.125, 0.25, 0.5, and 1.0 mg/mL). Simple linear regression models were calculated in GraphPad Prism software (version 8.4.2).

Quantification of hydrophobic small molecule encapsulation. DiI (λ_{Ex}=549 nm; λ_{Em}=565 nm) fluorescence was measured using a Shimadzu RF-6000 spectrofluorophotometer (Shimadzu). Curcumin loading was determined by measuring the absorbance of 450 nm light using a BioTek Synergy 2 plate reader (BioTek Instruments). Encapsulation efficiency was determined by comparing the fluorescence or absorbance before and after purification with an LH-20 column.

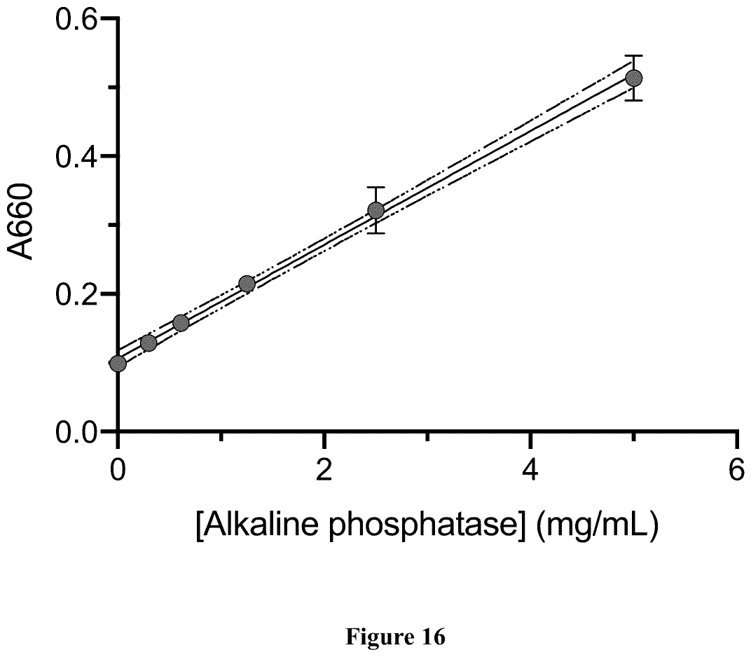

Hydrophilic biologic encapsulation and loading efficiency characterization. Hydrophilic encapsulation studies were performed using two hydrophilic cargoes: alkaline phosphatase (AP; Sigma Aldrich) and 70 kDa dextran-tetramethylrhodamine (Dex-TMR; Life Technologies). Briefly, 5 mg of AP and 100 μg of Dex-TMR were dissolved in 1 mL of PBS and this solution was used to rehydrate powder consisting of PEG-b-PPS PS polymer and mannitol carbohydrate prepared at a 1:3 ratio. The resulting nanocarriers were purified by size exclusion chromatography using a Sepharose 6B column. Dex-TMR (λ_{Ex}=555 nm; λ_{Em}=580 nm) loading efficiency was measured using a Shimadzu RF-6000 spectrofluorophotometer (Shimadzu). The protein concentration in alkaline phosphatase-loaded PS was determined using the Pierce A660 assay after disrupting the AP-encapsulating PS nanocarriers using 1% Triton-X. The Pierce A660 assay was calibrated against an AP concentration series (0, 0.3, 0.6125, 1.25, 2.5, 5.0 mg/mL AP) (FIG. 16). Absorbance was measured using a BioTek Synergy 2 plate reader (BioTek Instruments). Encapsulation efficiency was determined by comparing the fluorescence or absorbance before and after purification by SEC.

Characterization of enzyme bioactivity. After rehydrating polymersome-forming powders with aqueous media containing AP enzyme (5 mg/mL; Sigma), a fraction of the formulation was purified by SEC as described elsewhere in this methods section. Encapsulated AP enzyme was released by treating nanoparticle suspensions with 1% Triton-X. Samples were incubated with 5-bromo-4-chloro-3-indolyl phosphate (BCIP)/nitro blue tetrazolium (NBT) substrate (Sigma-Aldrich), and enzyme activity was monitored over a 75-minute time interval by measuring the absorbance of 630 nm light using a BioTek Synergy 2 plate reader (BioTek Instruments).

Macrophage cell culture. RAW 264.7 macrophages were purchased from ATCC and were cultured in T75 polystyrene tissue culture flasks (BD Biosciences) in Dulbecco's Modified Eagle Medium supplemented with 10% fetal bovine serum (FBS) (Gibco) and 1% penicillin/streptomycin antibiotics (Life Technologies). Cells were passaged by mechanical scraping after reaching 70-80% confluency.

Primary cell culture. Human umbilical vein endothelial cells (HUVECs) were purchased from Lonza, Ltd. The HUVECs were cultured in endothelial cell growth basal medium-2 (EBM-2; Lonza) that was supplemented with an EGM-2 BulletKit (Lonza) and FBS (Gibco). All cells were cultured at 37° C., 5% CO₂ in T25 or T75 flasks. Media was replaced every two days. Cells were passaged by trypsinization.

MTT assay. The MTT (3-(4,5-dimethylthiazolyl-2)-2,5-diphenyltetrazolium bromide) assay⁵² was performed on cells (n=5/condition) treated with nanocarriers (0.02, 0.2, or 2.0 mg/mL polymer concentration), mannitol (3 mg/mL; Sigma), or Carboplatin (1 mg/mL; Sigma) for 24 h at 37° C., 5.0% CO₂. Cells were treated with 0.5 mg/mL thiazolyl blue tetrazolium bromide (MTT) (Sigma) and viability was assessed after 5 h. The absorbance of 570 nm light was measured using a BioTek Synergy 2 plate reader (BioTek Instruments). The percentage of viable cells was calculated by dividing the absorbance of the experimental group by that of the mean fluorescence of PBS-treated cells.

Flow cytometric analysis of nanocarrier uptake and cytotoxicity. For each specified cell line, a total of 100,000 cells were seeded per well in 24-well polystyrene plates (Falcon). Cells were allowed to adhere overnight at 37° C., 4.0% CO₂. For uptake studies involving endocytosis inhibitor pre-treatments, cells were incubated with either PBS (i.e., no inhibitor), cytochalasin D (CytD, 50 μM) phagocytosis inhibitor, or chlorpromazine (CPZ, 50 M) clathrin-mediated endocytosis inhibitor prior to administering nanocarriers. Inhibitor concentrations were chosen based on published reports^{36,53}. Cells were treated with nanocarriers at the specified polymer concentration for 2 h. All cellular incubations were performed at 37° C., 5.0% CO₂. After the nanocarrier treatment period, cells were harvested by either mechanical scraping (macrophages) or trypsinization (HUVECs). Cells were stained with fixable Zombie Aqua viability dye (Biolegend) for 20 min at 4° C. to assess cytotoxicity via flow cytometry. A BD LSRFortessa 6-Laser Flow Cytometer was used to perform flow cytometry. A total of 10,000 single cell events were recorded per sample and the resulting data were analyzed using the Cytobank analysis suite⁵⁴. The median fluorescence intensity (MFI) was normalized by subtracting the average MFI from untreated cells and was used to quantify the cellular uptake of nanocarriers.

Statistical analysis. Statistical analyses were performed using GraphPad Prism software (version 9.0.0).

### REFERENCES

1 S. Matoori, Y. Bao, A. Schmidt, E. J. Fischer, R. Ochoa-Sanchez, M. Tremblay, M. M. Oliveira, C. F. Rose and J.-C. Leroux, Small, 2019, 15, e1902347.
2 T. Stack, A. Vahabikashi, M. Johnson and E. Scott, J Biomed Mater Res A, 2018, 106, 1771-1779.
3 N. B. Karabin, S. Allen, H.-K. Kwon, S. Bobbala, E. Firlar, T. Shokuhfar, K. R. Shull and E. A. Scott, Nature Communications, DOI: 10.1038/s41467-018-03001-9.
4 S. Yi, N. B. Karabin, J. Zhu, S. Bobbala, H. Lyu, S. Li, Y. Liu, M. Frey, M. Vincent and E. A. Scott, Front. Bioeng. Biotechnol., DOI: 10.3389/fbioe.2020.00542.
5 B. K. Johnson and R. K. Prud'homme, AIChE J, 2003, 49, 2264-2282.
6 W. S. Saad and R. K. Prud'homme, Nano Today, 2016, 11, 212-227.
7 S. Allen, O. Osorio, Y.-G. Liu and E. Scott, J Control Release, 2017, 262, 91-103.
8 S. D. Allen, S. Bobbala, N. B. Karabin, M. Modak and E. A. Scott, ACS Appl Mater Interfaces, 2018, 10, 33857-33866.
9 S. Bobbala, S. D. Allen and E. A. Scott, Nanoscale, 2018, 10, 5078-5088.
10 S. Allen, M. Vincent and E. Scott, J Vis Exp, 2018, e57793.
11 S. K. R. Bobbala and P. R. Veerareddy, J Liposome Res, 2012, 22, 285-294.
12 V. Nekkanti, Z. Wang and G. V. Betageri, AAPS PharmSciTech, 2016, 17, 851-862.
13 K. Y. Janga, R. Jukanti, A. Velpula, S. Sunkavalli, S. Bandari, P. Kandadi and P. R. Veerareddy, Eur J Pharm Biopharm, 2012, 80, 347-357.
14 P. R. Veerareddy and S. K. R. Bobbala, Drug Dev Ind Pharm, 2013, 39, 909-917.
15 A. Gurrapu, R. Jukanti, S. R. Bobbala, S. Kanuganti and J. B. Jeevana, Advanced Powder Technology, 2012, 23, 583-590.
16 V. Nekkanti, N. Venkatesan and G. V. Betageri, Curr Pharm Biotechnol, 2015, 16, 303-312.
17 S. Yi, S. D. Allen, Y.-G. Liu, B. Z. Ouyang, X. Li, P. Augsornworawat, E. B. Thorp and E. A. Scott, ACS Nano, 2016, 10, 11290-11303.
18 M. P. Vincent, S. Bobbala, N. B. Karabin, M. Frey, Y. Liu, J. O. Navidzadeh, T. Stack and E. A. Scott, Nat Commun, 2021, 12, 648.
19 A. Napoli, M. Valentini, N. Tirelli, M. Müller and J. A. Hubbell, Nature Materials, 2004, 3, 183-189.
20 A. Napoli, M. J. Boerakker, N. Tirelli, R. J. M. Nolte, N. A. J. M. Sommerdijk and J. A. Hubbell, Langmuir, 2004, 20, 3487-3491.
21 A. E. Vasdekis, E. A. Scott, C. P. O'Neil, D. Psaltis and Jeffrey. A. Hubbell, ACS Nano, 2012, 6, 7850-7857.
22 S. Bobbala, S. D. Allen, S. Yi, M. Vincent, M. Frey, N. B. Karabin and E. A. Scott, Nanoscale, 2020, 12, 5332-5340.
23 N. B. Karabin, M. P. Vincent, S. D. Allen, S. Bobbala, M. A. Frey, S. Yi, Y. Yang and E. A. Scott, bioRxiv, 2020, 2020.09.02.280404.
24 E. A. Scott, A. Stano, M. Gillard, A. C. Maio-Liu, M. A. Swartz and J. A. Hubbell, Biomaterials, 2012, 33, 6211-6219.
25 S. D. Allen, Y.-G. Liu, S. Bobbala, L. Cai, P. I. Hecker, R. Temel and E. A. Scott, Nano Res., 2018, 11, 5689-5703.
26 S. Cerritelli, C. P. O'Neil, D. Velluto, A. Fontana, M. Adrian, J. Dubochet and J. A. Hubbell, Langmuir, 2009, 25, 11328-11335.
27 H. L. Ohrem, E. Schornick, A. Kalivoda and R. Ognibene, Pharm Dev Technol, 2014, 19, 257-262.
28 M. K. Lee, M. Y. Kim, S. Kim and J. Lee, Journal of Pharmaceutical Sciences, 2009, 98, 4808-4817.
29 Y. Cui, P. Cui, B. Chen, S. Li and H. Guan, Drug Development and Industrial Pharmacy, 2017, 43, 519-530.
30 B. M. Discher, Y.-Y. Won, D. S. Ege, J. C.-M. Lee, F. S. Bates, D. E. Discher and D. A. Hammer, Science, 1999, 284, 1143-1146.
31 K. Izutsu, S. Yoshioka and T. Terao, Pharm Res, 1993, 10, 1232-1237.
32 K. Izutsu, S. Yoshioka and S. Kojima, Pharm Res, 1994, 11, 995-999.
33 K. Izutsu, S. Yoshioka and T. Terao, Chem Pharm Bull (Tokyo), 1994, 42, 5-8.
34 T. Stack, M. Vincent, A. Vahabikashi, G. Li, K. M. Perkumas, W. D. Stamer, M. Johnson and E. Scott, Small, 2020, 2004205.
35 M. R. Jackman, W. Shurety, J. A. Ellis and J. P. Luzio, Journal of Cell Science, 1994, 107, 2547-2556.
36 L. Sasso, L. Purdie, A. Grabowska, A. T. Jones and C. Alexander, Journal of Interdisciplinary Nanomedicine, 2018, 3, 67-81.
37 S. Yi, X. Zhang, M. H. Sangji, Y. Liu, S. D. Allen, B. Xiao, S. Bobbala, C. L. Braverman, L. Cai, P. I. Hecker, M. DeBerge, E. B. Thorp, R. E. Temel, S. I. Stupp and E. A. Scott, Adv Funct Mater, 2019, 29, 1904399.
38 S. J. Bogdanowich-Knipp, S. Chakrabarti, T. D. Williams, R. K. Dillman and T. J. Siahaan, J Pept Res, 1999, 53, 530-541.
39 S. J. Bogdanowich-Knipp, D. S. Jois and T. J. Siahaan, J Pept Res, 1999, 53, 523-529.
40 M. Amin, A. Badiee and M. R. Jaafari, Int J Pharm, 2013, 458, 324-333.
41 P. K. Dubey, V. Mishra, S. Jain, S. Mahor and S. P. Vyas, J Drug Target, 2004, 12, 257-264.
42 Z. Song, Y. Lin, X. Zhang, C. Feng, Y. Lu, Y. Gao and C. Dong, Int J Nanomedicine, 2017, 12, 1941-1958.
43 M. D. Pierschbacher and E. Ruoslahti, Nature, 1984, 309, 30-33.
44 J. P. Kim, K. Zhang, R. H. Kramer, T. J. Schall and D. T. Woodley, J Invest Dermatol, 1992, 98, 764-770.
45 E. Ruoslahti and M. D. Pierschbacher, Cell, 1986, 44, 517-518.
46 K. M. Yamada and D. W. Kennedy, J Cell Biochem, 1985, 28, 99-104.
47 S. Zitzmann, V. Ehemann and M. Schwab, Cancer Res, 2002, 62, 5139-5143.
48 S. Cressman, Y. Sun, E. J. Maxwell, N. Fang, D. D. Y. Chen and P. R. Cullis, Int J Pept Res Ther, 2009, 15, 49-59.
49 K. M. Holtz and E. R. Kantrowitz, FEBS Letters, 1999, 462, 7-11.
50 F. Ahmed and D. E. Discher, Journal of Controlled Release, 2004, 96, 37-53.
51 M. D. Shin, S. Shukla, Y. H. Chung, V. Beiss, S. K. Chan, O. A. Ortega-Rivera, D. M. Wirth, A. Chen, M. Sack, J. K. Pokorski and N. F. Steinmetz, Nat Nanotechnol, 2020, 15, 646-655.
52 T. Mosmann, J. Immunol. Methods, 1983, 65, 55-63.
53 Y. Wei, T. Tang and H.-B. Pang, Nature Communications, 2019, 10, 3646.
53 T. J. Chen and N. Kotecha, Curr. Top. Microbiol. Immunol, 2014, 377, 127-157.

1. A rehydratable powdered formulation comprising:
a. a polymeric amphiphile; and
b. a carbohydrate.

2. The formulation of claim 1, wherein the polymeric amphiphile is selected from a group consisting of poly(ethylene glycol)-block-poly(propylene sulfide) (PEG-b-PPS) copolymer, PEG-b-polystyrene polymer, and PEG-b-polycaprolactone polymer.

3. The formulation of claim 1, wherein the polymeric amphiphile has a glass transition temperature lower than −30° C.

4. The formulation of claim 1, wherein the polymeric amphiphile coats the carbohydrate.

5. (canceled)

6. The formulation of claim 1, wherein the carbohydrate is selected from a group consisting of mannitol, trehalose, lactose, and glucose.

7. The formulation of claim 1, wherein the formulation further comprises a ligand comprising a peptide, a PEG spacer, and a lipid anchor.

8. The formulation of claim 7, wherein the PEG spacer has 6 units and the lipid anchor is derived from palmitoleic acid.

9. (canceled)

10. The formulation of claim 7, wherein the peptide and the polymeric amphiphile has a molar ratio of 1%-5%.

11. The formulation of claim 1, wherein the polymeric amphiphile is PEG-b-PPS copolymer that has a PEG weight fraction range of 0.2-0.6.

12. The formulation of claim 1, wherein the formulation further comprises a hydrophobic cargo.

13. (canceled)

14. A method of producing an aqueous suspension of a nanocarrier from a rehydratable powdered formulation comprising a polymeric amphiphile and a carbohydrate, the method comprising:
a. contacting the formulation with a liquid to form a rehydrated mixture; and
b vortexing the rehydrated mixture.

15. The method of claim 14, wherein the polymeric amphiphile is PEG-b-polycaprolactone and the method further comprises a step following step (a), the step comprising heating the rehydrated mixture at 60° C. in a water bath.

16. (canceled)

17. The method of claim 14, wherein the liquid further comprises a hydrophilic cargo and/or a ligand.

18. The method of claim 14, wherein the formulation further comprises a hydrophobic cargo and/or ligand, and the nanocarrier has a loading efficiency of greater than 95% of the hydrophobic cargo and/or the ligand.

19. A method of producing a rehydratable powdered formulation, the method comprising:
(a) dissolving a polymeric amphiphile in a first organic solvent to form a solution;
(b) adding to the solution a carbohydrate to make a mixture; and
(c) evaporating the organic solvent from the mixture in a vacuum desiccator to form the rehydratable powder comprising a polymeric amphiphile and carbohydrate.

20. The method of claim 19, wherein the method further comprises a step following step (b), the step comprising adding a solution comprising a hydrophobic cargo and/or a ligand and a second organic solvent to the mixture prior to step (c).

21. The method of claim 19, wherein the carbohydrate is selected from a group consisting of mannitol, trehalose, lactose, and glucose.

22. The method of claim 19, wherein the first organic solvent is selected from a group consisting of dichloromethane, acetone, chloroform, and tetrahydrofuran.

23. (canceled)

24. The method of claim 19, wherein the polymeric amphiphile is selected from a group consisting of poly(ethylene glycol)-block-poly(propylene sulfide) (PEG-b-PPS) copolymer, PEG-b-polystyrene polymer, and PEG-b-polycaprolactone polymer.

25. The method of claim 19, wherein the polymeric amphiphile is PEG-b-PPS copolymer that has a PEG weight fraction range of 0.2-0.6.
